(19) United States
(12) Patent Application Publication
(10) Pub. No.: US 2016/0002250 A1
(43) Pub. Date: Jan. 7, 2016

(54) POLYMORPHS AND SALTS OF A COMPOUND

(71) Applicant: FORUM Pharmaceuticals Inc., Waltham, MA (US)

(72) Inventor: Patricia OLIVER, Waltham, MA (US)

(21) Appl. No.: 14/851,250

(22) Filed: Sep. 11, 2015

Related U.S. Application Data
(63) Continuation of application No. PCT/US14/26988, filed on Mar. 14, 2014.
(60) Provisional application No. 61/785,692, filed on Mar. 14, 2013.

(51) Int. Cl. C07D 487/04

(52) U.S. Cl. CPC C07D 487/04; C07B 2200/13

(57) ABSTRACT

Disclosed are novel crystalline polymorphic forms of 4-(4-(imidazo[1,2-b]pyridazin-2-ylmethoxy)phenyl)-2,2-dimethyl-5-(pyridin-4-yl)furan-3(2H)-one and salts thereof, methods of preparing the crystalline polymorphic forms and salts thereof, pharmaceutical compositions comprising the crystalline polymorphic forms and salts thereof, and methods of treating CNS disorders, eating disorders, obesity, compulsive gambling, sexual disorders, narcolepsy, sleep disorders, diabetes, metabolic syndrome, schizophrenia, schizo-affective conditions, Huntington's disease, bipolar disorders, dystonic conditions and tardive dyskinesia, or for use in smoking cessation treatment in a patient using the crystalline polymorphic forms and salts thereof.

### CROSS-REFERENCE TO RELATED APPLICATION

This application is a continuation of International Application PCT/US2014/026988 filed Mar. 14, 2014, which claims priority to U.S. Provisional Patent Application Ser. No. 61/785,692, filed Mar. 14, 2013, the entire contents of which are incorporated by reference herein in their entirety.

### FIELD OF THE INVENTION

The invention generally relates to the field of crystalline polymorphs and salts of phosphodiesterase (PDE) inhibitors and more specifically to novel polymorphic forms of 4-(4-(imidazo[1,2-b]pyridazin-2-ylmethoxy)phenyl)-2,2-dimethyl-5-(pyridin-4-yl)furan-3(2H)-one or salts thereof.

### BACKGROUND OF THE INVENTION

Cyclic phosphodiesterases are intracellular enzymes which, through the hydrolysis of cyclic nucleotides cAMP and cGMP, regulate the levels of these mono phosphate nucleotides which serve as second messengers in the signaling cascade of G-protein coupled receptors. In neurons, PDEs also play a role in the regulation of downstream cGMP and cAMP dependent kinases which phosphorylate proteins involved in the regulation of synaptic transmission and homeostasis. To date, eleven different PDE families have been identified which are encoded by 21 genes. The PDEs contain a variable N-terminal regulatory domain and a highly conserved C-terminal catalytic domain and differ in their substrate specificity, expression and localization in cellular and tissue compartments, including the CNS.

The discovery of a new PDE family, PDE10, was reported simultaneously by three groups in 1999 (Soderling et al., “Isolation and characterization of a dual-substrate phosphodiesterase gene family: PDE10A” Proc. Natl Sci. 1999, 96, 7071-7076; Loughney et al., “Isolation and characterization of PDE10A, a novel human 3′, 5′-cyclic nucleotide phosphodiesterase” Gene 1999, 234, 109-117; Fujishige et al., “Cloning and characterization of a novel human phosphodiesterase that hydrolyzes both cAMP and cGMP (PDE10A)” J. Biol. Chem. 1999, 274, 18438-18445). The human PDE10 sequence is highly homologous to both the rat and mouse variants with 95% amino acid identity overall, and 98% identity conserved in the catalytic region.

PDE10 is primarily expressed in the brain (caudate nucleus and putamen) and is highly localized in the medium spiny neurons of the striatum, which is one of the principal inputs to the basal ganglia. This localization of PDE10 has led to speculation that it may influence the dopaminergic and glutamatergic pathways both which play roles in the pathology of various psychotic and neurodegenerative disorders.

PDE10 hydrolyzes both cAMP (Kₘ=0.05 uM) and cGMP (Kₘ=3 uM) (Soderling et al., “Isolation and Characterization of a dual-substrate phosphodiesterase gene family: PDE10.” Proc. Natl Sci. USA 1999, 96(12), 7071-7076). In addition, PDE10 has a five-fold greater Vₘₐₓ for cGMP than for cAMP and these in vitro kinetic data have led to the speculation that PDE10 may act as a cAMP-inhibited cGMP phosphodiesterase in vivo (Soderling and Beavo, “Regulation of cAMP and cGMP signaling: New phosphodiesterases and new functions,” Curr. Opin. Cell Biol., 2000, 12, 174-179).

PDE10 is also one of five phosphodiesterase members to contain a tandem GAF domain at their N-terminus. It is differentiated by the fact that the other GAF containing PDEs (PDE2, 5, 6, and 11) bind cGMP while recent data points to the tight binding of cAMP to the GAF domain of PDE10 (Handa et al., “Crystal structure of the GAF-B domain from human phosphodiesterase 10A complexed with its ligand, cAMP” J. Biol. Chem. 2008, May 13^{th}, ePub).

PDE10 inhibitors have been disclosed for the treatment of a variety of neurological and psychiatric disorders including Parkinson's disease, schizophrenia, Huntington's disease, delusional disorders, drug-induced psychoses, obsessive compulsive and panic disorders (US Patent Application 2003/0032579). Studies in rats (Kostowski et al., “Papaverine drug induced stereotypy and catalepsy and biogenic amines in the brain of the rat” Pharmacol. Biochem. Behav. 1976, 5, 15-17) have showed that papaverine, a selective PDE10 inhibitor, reduces apomorphine induced stereotypies and rat brain dopamine levels and increases haloperidol induced catalepsy. This experiment lends support to the use of a PDE10 inhibitor as an antipsychotic since similar trends are seen with known, marketed antipsychotics.

Antipsychotic medications are the mainstay of current treatment for schizophrenia. Conventional or classic antipsychotics, typified by haloperidol, were introduced in the mid-1950s and have a proven track record over the last half century in the treatment of schizophrenia. While these drugs are effective against the positive, psychotic symptoms of schizophrenia, they show little benefit in alleviating negative symptoms or the cognitive impairment associated with the disease. In addition, drugs such as haloperidol have extreme side effects such as extrapyramidal symptoms (EPS) due to their specific dopamine D2 receptor interaction. An even more severe condition characterized by significant, prolonged, abnormal motor movements known as tardive dyskinesia also may emerge with prolonged classic antipsychotic treatment.

The 1990s saw the development of several new drugs for schizophrenia, referred to as atypical antipsychotics, typified by risperidone and olanzapine and most effectively, clozapine. These atypical antipsychotics are generally characterized by effectiveness against both the positive and negative symptoms associated with schizophrenia, but have little effectiveness against cognitive deficiencies and persisting cognitive impairment remains a serious public health concern (Davis et al., “Dose response and dose equivalence of antipsychotics.” Journal of Clinical Psychopharmacology, 2004, 24 (2), 192-208; Friedman et al., “Treatment of psychosis in Parkinson's disease: Safety considerations.” Drug Safety, 2003, 26 (9), 643-659). In addition, the atypical antipsychotic agents, while effective in treating the positive and, to some degree, negative symptoms of schizophrenia, have significant side effects. For example, clozapine which is one of the most clinically effective antipsychotic drugs, shows agranulocytosis in approximately 1.5% of patients with fatalities due to this side effect being observed. Other atypical antipsychotic drugs have significant side effects including metabolic side effects (type 2 diabetes, significant weight gain, and dyslipidemia), sexual dysfunction, sedation, and potential cardiovascular side effects, that compromise their clinically effectiveness. In the large, recently published NIH sponsored CATIE study (Lieberman et al., “The Clinical Antipsychotic Trials Of Intervention Effectiveness (CATIE) Schizophrenia Trial: clinical comparison of subgroups with and without the metabolic syndrome.” Schizophrenia Research, 2005, 80 (1), 9-43), 74% of patients discontinued use of their antipsychotic medication within 18 months due to a number of factors including poor tolerability or incomplete efficacy. Therefore, a substantial clinical need still exists for more effective and better tolerated antipsychotic medications possibly through the use of PDE10 inhibitors.

4-(4-(imidazo[1,2-b]pyridazin-2-ylmethoxy)phenyl)-2,2-dimethyl-5-(pyridin-4-yl)furan-3(2H)-one (“Free Base”) is a PDE 10 inhibitors described in U.S. Pat. No. 8,343,973, filed on Dec. 18, 2009, the content of which is incorporated by reference herein.

There is a need to develop new salts and polymorphs of 4-(4-(imidazo[1,2-b]pyridazin-2-ylmethoxy)phenyl)-2,2-dimethyl-5-(pyridin-4-yl)furan-3(2H)-one that have acceptable solubility and bioavailability properties for formulation and administration as active pharmaceutical agents.

### SUMMARY OF THE INVENTION

The present invention is based, in part, upon the surprising discovery that certain crystalline polymorphs and/or salts of 4-(4-(imidazo[1,2-b]pyridazin-2-ylmethoxy)phenyl)-2,2-dimethyl-5-(pyridin-4-yl)furan-3(2H)-one possess desirable physical and chemical properties for formulation and administration as drugs for human therapy.

It is understood that any of the embodiments described below can be combined in any desired way, and any embodiment or combination of embodiments can be applied to each of the aspects described below, unless the context indicates otherwise.

These and other aspects and embodiments of the invention will be apparent to one of ordinary skill in the art based upon the following detailed description of the invention.

In one aspect, the invention provides crystalline 4-(4-(imidazo[1,2-b]pyridazin-2-ylmethoxy)phenyl)-2,2-dimethyl-5-(pyridin-4-yl)furan-3(2H)-one having a peak position at about 6.7, 10.8, 15.8, 18.0, 19.4, 20.2, 21.1, 21.5, or 28.8 degrees 2-theta in an x-ray powder diffraction pattern obtained using Cu-Kα radiation.

In some embodiments, the crystalline 4-(4-(imidazo[1,2-b]pyridazin-2-ylmethoxy)phenyl)-2,2-dimethyl-5-(pyridin-4-yl)furan-3(2H)-one has a peak position at about 6.7, 10.8, 18.0, 19.4, 21.1, or 21.5 degrees 2-theta in an x-ray powder diffraction pattern obtained using Cu-Kα radiation.

In some embodiments, the crystalline 4-(4-(imidazo[1,2-b]pyridazin-2-ylmethoxy)phenyl)-2,2-dimethyl-5-(pyridin-4-yl)furan-3(2H)-one is characterized by the X-ray powder diffractogram substantially as shown in FIG. 2, Form 2, when measured at room temperature using Cu-Kα radiation.

In some embodiments, the crystalline 4-(4-(imidazo[1,2-b]pyridazin-2-ylmethoxy)phenyl)-2,2-dimethyl-5-(pyridin-4-yl)furan-3(2H)-one is characterized by respective lattice parameters, a, b, and c of about 11.9 Å, 18.0 Å, and 19.4 Å, respectively, and β of about 102.8° in the monoclinic crystal system P2₁ space group, when measured with Cu-Kα radiation at about 100 K.

In some embodiments, the crystalline 4-(4-(imidazo[1,2-b]pyridazin-2-ylmethoxy)phenyl)-2,2-dimethyl-5-(pyridin-4-yl)furan-3(2H)-one is at least about 95% chemically pure.

In some embodiments, the crystalline 4-(4-(imidazo[1,2-b]pyridazin-2-ylmethoxy)phenyl)-2,2-dimethyl-5-(pyridin-4-yl)furan-3(2H)-one is at least about 97% chemically pure.

In some embodiments, the crystalline 4-(4-(imidazo[1,2-b]pyridazin-2-ylmethoxy)phenyl)-2,2-dimethyl-5-(pyridin-4-yl)furan-3(2H)-one is at least about 99% chemically pure.

In some embodiments, the crystalline purity of 4-(4-(imidazo[1,2-b]pyridazin-2-ylmethoxy)phenyl)-2,2-dimethyl-5-(pyridin-4-yl)furan-3(2H)-one is determined by HPLC.

In some embodiments, the crystalline 4-(4-(imidazo[1,2-b]pyridazin-2-ylmethoxy)phenyl)-2,2-dimethyl-5-(pyridin-4-yl)furan-3(2H)-one has an endothermic onset at about 140-165° C. in a differential scanning calorimetry (DSC) profile.

In some embodiments, the crystalline 4-(4-(imidazo[1,2-b]pyridazin-2-ylmethoxy)phenyl)-2,2-dimethyl-5-(pyridin-4-yl)furan-3(2H)-one has an endothermic onset at about 145° C. in a differential scanning calorimetry (DSC) profile.

In some embodiments, the crystalline 4-(4-(imidazo[1,2-b]pyridazin-2-ylmethoxy)phenyl)-2,2-dimethyl-5-(pyridin-4-yl)furan-3(2H)-one has an endothermic onset at about 185° C. in a differential scanning calorimetry (DSC) profile.

In some embodiments, the crystalline 4-(4-(imidazo[1,2-b]pyridazin-2-ylmethoxy)phenyl)-2,2-dimethyl-5-(pyridin-4-yl)furan-3(2H)-one is characterized by the differential scanning calorimetry (DSC) profile substantially as shown in FIG. 8.

In some embodiments, the crystalline 4-(4-(imidazo[1,2-b]pyridazin-2-ylmethoxy)phenyl)-2,2-dimethyl-5-(pyridin-4-yl)furan-3(2H)-one is stable at room temperature under air for at least about 4, 6, 8, 10, 12, or 20 weeks.

In another aspect, the invention provides crystalline 4-(4-(imidazo[1,2-b]pyridazin-2-ylmethoxy)phenyl)-2,2-dimethyl-5-(pyridin-4-yl)furan-3(2H)-one having a peak position at about 7.9, 8.0, 10.2, 13.7, 14.0, 16.2, 17.6, 19.1, 19.3, 21.2, or 21.4 degrees 2-theta in an x-ray powder diffraction pattern obtained using Cu-Kα radiation.

In some embodiments, the crystalline 4-(4-(imidazo[1,2-b]pyridazin-2-ylmethoxy)phenyl)-2,2-dimethyl-5-(pyridin-4-yl)furan-3(2H)-one has a peak position at about 19.1, 19.3, 21.2, or 21.4 degrees 2-theta in an x-ray powder diffraction pattern obtained using Cu-Kα radiation.

In some embodiments, the crystalline 4-(4-(imidazo[1,2-b]pyridazin-2-ylmethoxy)phenyl)-2,2-dimethyl-5-(pyridin-4-yl)furan-3(2H)-one is characterized by the X-ray powder diffractogram substantially as shown in FIG. 2, Form 1, when measured at room temperature using Cu-Kα radiation.

In some embodiments, the crystalline 4-(4-(imidazo[1,2-b]pyridazin-2-ylmethoxy)phenyl)-2,2-dimethyl-5-(pyridin-4-yl)furan-3(2H)-one is characterized by respective lattice parameters, a, b, and c of about 12.2 Å, 27.4 Å, and 12.4 Å, respectively, and β of about 96.7° in the monoclinic crystal system P2₁ space group, when measured with Cu-Kα radiation at about 120 K.

In some embodiments, the crystalline 4-(4-(imidazo[1,2-b]pyridazin-2-ylmethoxy)phenyl)-2,2-dimethyl-5-(pyridin-4-yl)furan-3(2H)-one is at least about 95% chemically pure.

In some embodiments, the crystalline 4-(4-(imidazo[1,2-b]pyridazin-2-ylmethoxy)phenyl)-2,2-dimethyl-5-(pyridin-4-yl)furan-3(2H)-one is at least about 97% chemically pure.

In some embodiments, the crystalline 4-(4-(imidazo[1,2-b]pyridazin-2-ylmethoxy)phenyl)-2,2-dimethyl-5-(pyridin-4-yl)furan-3(2H)-one is at least about 99% chemically pure.

In some embodiments, the purity of crystalline 4-(4-(imidazo[1,2-b]pyridazin-2-ylmethoxy)phenyl)-2,2-dimethyl-5-(pyridin-4-yl)furan-3(2H)-one is determined by HPLC.

In some embodiments, the crystalline 4-(4-(imidazo[1,2-b]pyridazin-2-ylmethoxy)phenyl)-2,2-dimethyl-5-(pyridin-4-yl)furan-3(2H)-one has an endothermic onset at about 180-190° C. in a differential scanning calorimetry (DSC) profile.

In some embodiments, the crystalline 4-(4-(imidazo[1,2-b]pyridazin-2-ylmethoxy)phenyl)-2,2-dimethyl-5-(pyridin-4-yl)furan-3(2H)-one has an endothermic onset at about 185-186° C. in a differential scanning calorimetry (DSC) profile.

In some embodiments, the crystalline 4-(4-(imidazo[1,2-b]pyridazin-2-ylmethoxy)phenyl)-2,2-dimethyl-5-(pyridin-4-yl)furan-3(2H)-one has a melting point of about 185-186° C.

In some embodiments, the crystalline 4-(4-(imidazo[1,2-b]pyridazin-2-ylmethoxy)phenyl)-2,2-dimethyl-5-(pyridin-4-yl)furan-3(2H)-one has a melting point of about 185° C.

In some embodiments, the crystalline 4-(4-(imidazo[1,2-b]pyridazin-2-ylmethoxy)phenyl)-2,2-dimethyl-5-(pyridin-4-yl)furan-3(2H)-one is characterized by the differential scanning calorimetry (DSC) profile substantially as shown in FIG. 5.

In some embodiments, the crystalline 4-(4-(imidazo[1,2-b]pyridazin-2-ylmethoxy)phenyl)-2,2-dimethyl-5-(pyridin-4-yl)furan-3(2H)-one is stable at room temperature under air for at least about 4, 6, 8, 10, 12, or 20 weeks.

In yet another aspect, the invention provides a pharmaceutical composition comprising the crystalline 4-(4-(imidazo[1,2-b]pyridazin-2-ylmethoxy)phenyl)-2,2-dimethyl-5-(pyridin-4-yl)furan-3(2H)-one of any one of embodiments described herein and a pharmaceutically acceptable excipient.

In some embodiments, the pharmaceutical composition comprises the crystalline 4-(4-(imidazo[1,2-b]pyridazin-2-ylmethoxy)phenyl)-2,2-dimethyl-5-(pyridin-4-yl)furan-3(2H)-one in a therapeutically effective amount.

In some embodiments, the pharmaceutical composition is formulated for oral administration.

In some embodiments, the pharmaceutical composition is in the form of a unit dosage.

In some embodiments, the pharmaceutical composition is in the form of a tablet, a capsule or a powder.

In some embodiments, the pharmaceutical composition is in the form of a tablet.

In yet another aspect, the invention provides a method of treating CNS disorders, eating disorders, obesity, compulsive gambling, sexual disorders, narcolepsy, sleep disorders, diabetes, metabolic syndrome, schizophrenia, schizo-affective conditions, Huntington's disease, bipolar disorders, dystonic conditions and tardive dyskinesia, or for use in smoking cessation treatment in a patient, the method comprising administering to the patient in need thereof an effective amount of the crystalline 4-(4-(imidazo[1,2-b]pyridazin-2-ylmethoxy)phenyl)-2,2-dimethyl-5-(pyridin-4-yl)furan-3(2H)-one of any one of the embodiments described herein.

In some embodiments, the patient is a mammal.

In some embodiments, the mammal is a human.

In some embodiments, the crystalline 4-(4-(imidazo[1,2-b]pyridazin-2-ylmethoxy)phenyl)-2,2-dimethyl-5-(pyridin-4-yl)furan-3(2H)-one is orally administered.

In some embodiments, the crystalline 4-(4-(imidazo[1,2-b]pyridazin-2-ylmethoxy)phenyl)-2,2-dimethyl-5-(pyridin-4-yl)furan-3(2H)-one is administered once or twice daily.

In some embodiments, the crystalline 4-(4-(imidazo[1,2-b]pyridazin-2-ylmethoxy)phenyl)-2,2-dimethyl-5-(pyridin-4-yl)furan-3(2H)-one is administered as a tablet or a capsule.

In some embodiments, the method is for treating Huntington's disease.

In some embodiments, the method is for treating schizophrenia.

### BRIEF DESCRIPTION OF THE FIGURES

FIG. 1 illustrates the chemical structure of 4-(4-(imidazo[1,2-b]pyridazin-2-ylmethoxy)phenyl)-2,2-dimethyl-5-(pyridin-4-yl)furan-3(2H)-one, in which the basic nitrogen atoms N¹ and N² are identified.

FIG. 2 is a graphic representation of X-ray powder diffractograms of crystalline Form 1 and Form 2 of 4-(4-(imidazo[1,2-b]pyridazin-2-ylmethoxy)phenyl)-2,2-dimethyl-5-(pyridin-4-yl)furan-3(2H)-one.

FIG. 3 is a model representation of the molecular configuration of crystalline Form 1 of 4-(4-(imidazo[1,2-b]pyridazin-2-ylmethoxy)phenyl)-2,2-dimethyl-5-(pyridin-4-yl)furan-3(2H)-one.

FIG. 4 is a model representation of the crystal packing of crystalline Form 1 of 4-(4-(imidazo[1,2-b]pyridazin-2-ylmethoxy)phenyl)-2,2-dimethyl-5-(pyridin-4-yl)furan-3(2H)-one.

FIG. 5 is a graphic representation of a DSC thermogram (top panel) and a TGA thermogram (bottom panel) of crystalline Form 1 of 4-(4-(imidazo[1,2-b]pyridazin-2-ylmethoxy)phenyl)-2,2-dimethyl-5-(pyridin-4-yl)furan-3(2H)-one.

FIG. 6 is a model representation of the molecular configuration of crystalline Form 2 of 4-(4-(imidazo[1,2-b]pyridazin-2-ylmethoxy)phenyl)-2,2-dimethyl-5-(pyridin-4-yl)furan-3(2H)-one.

FIG. 7 is a model representation of the crystal packing of crystalline Form 2 of 4-(4-(imidazo[1,2-b]pyridazin-2-ylmethoxy)phenyl)-2,2-dimethyl-5-(pyridin-4-yl)furan-3(2H)-one.

FIG. 8 is a graphic representation of TGA and DSC thermograms of crystalline Form 2 of 4-(4-(imidazo[1,2-b]pyridazin-2-ylmethoxy)phenyl)-2,2-dimethyl-5-(pyridin-4-yl)furan-3(2H)-one using heat at 10° C./min under air or N₂.

FIG. 9 is a graphic representation of VT-XRPD of crystalline Form 2 of 4-(4-(imidazo[1,2-b]pyridazin-2-ylmethoxy)phenyl)-2,2-dimethyl-5-(pyridin-4-yl)furan-3(2H)-one.

FIG. 10 is a graphic representation of TGA and DSC thermograms of crystalline Form 2 of the Free Base in comparison with the single crystal of crystalline Form 2 of the Free Base.

FIG. 11 shows optical images corresponding to Hot Stage Microscopy of the crystalline Form 2 of the Free Base.

FIG. 12 shows optical images of the crystalline Form 1 of the Free Base obtained by slow cooling from toluene solution.

FIG. 13 illustrates the stability of amorphous Free Base after 24 h at elevated humidity monitored by X-ray powder diffractograms.

FIG. 14 is a graphic representation of X-ray powder diffractograms of crystalline solids obtained from maturation of amorphous 4-(4-(imidazo[1,2-b]pyridazin-2-ylmethoxy)phenyl)-2,2-dimethyl-5-(pyridin-4-yl)furan-3(2H)-one.

FIG. 15 is a graphic representation of X-ray powder diffractograms of crystalline solids obtained from competitive slurries.

FIG. 16 shows the optical images of single crystals of the crystalline Form 2 of the Free Base.

FIG. 17A shows the chemical purity of the crystalline Form 1 of the Free Base determined by HPLC.

FIG. 17B shows the chemical purity of the crystalline Form 2 of the Free Base determined by HPLC.

FIG. 18 shows the GVS analysis of the crystalline Form 1 of the Free Base (kinetic plot).

FIG. 19 shows the GVS analysis of the crystalline Form 1 of the Free Base (isotherm plot).

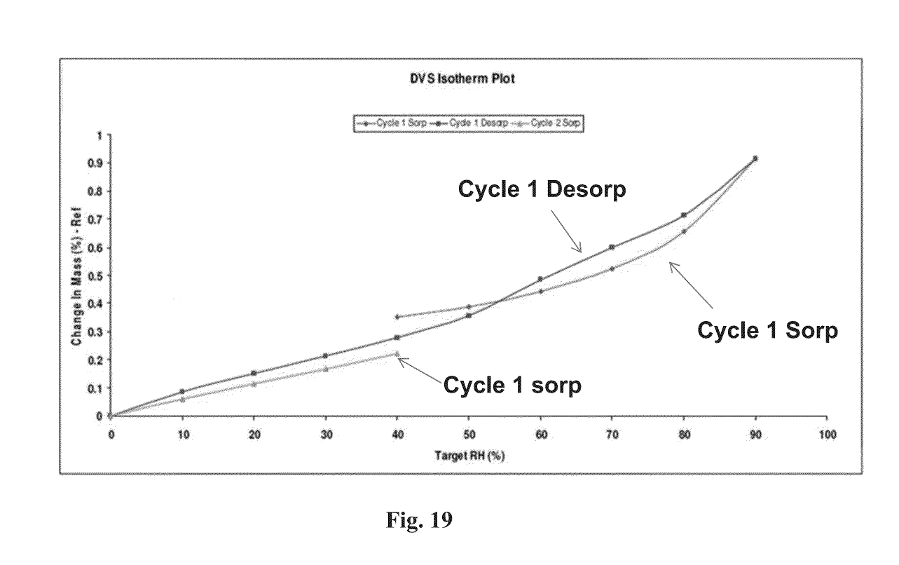

FIG. 20 shows the X-ray powder diffractograms of solids isolated from counter-ion screen of the Free Base in THF using one equivalent of acid.

FIG. 21 shows the X-ray powder diffractograms of solids isolated from counter-ion screen of the Free Base in MeOH using one equivalent of acid.

FIG. 22 shows the X-ray powder diffractograms of solids isolated from counter-ion screen of the Free Base using two equivalents of acid.

FIG. 23 shows the X-ray powder diffractograms of bis-HBr salt of the Free Base.

FIG. 24 shows the X-ray powder diffractograms of bis-Tosylate salt of the Free Base.

FIG. 25 shows the X-ray powder diffractograms of mono-Fumarate salt of the Free Base.

FIG. 26 shows the stability studies by X-ray powder diffractograms of various Free Base salts.

FIG. 27 shows the stability studies by X-ray powder diffractograms of the Free Base bis-tosylate salt.

FIG. 28 shows the X-ray powder diffractograms of the Free Base bis-mesylate salt.

FIG. 29 shows the X-ray powder diffractograms of the Free Base bis-tosylate salt isolated from various solvents.

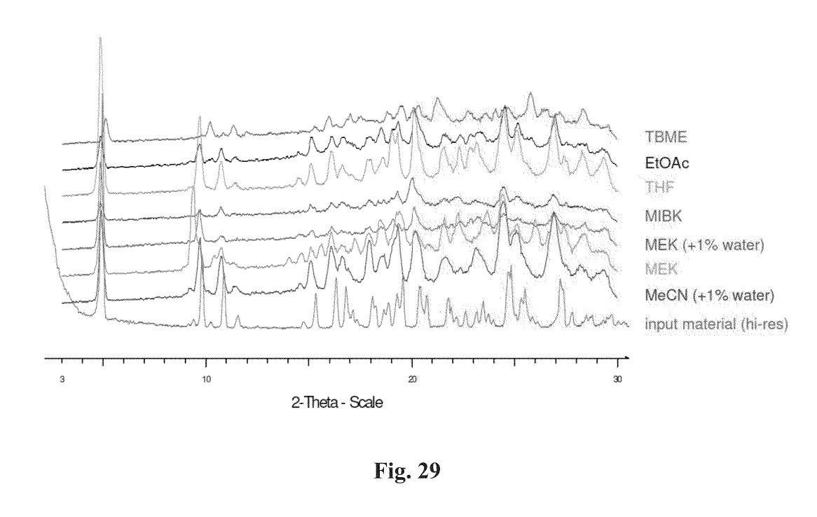

### DETAILED DESCRIPTION OF THE INVENTION

### 1. References and Definitions

The patent and scientific literature referred to herein establishes knowledge that is available to those of skill in the art. The issued U.S. patents, allowed U.S. applications, published U.S. applications, published foreign applications, foreign patents, and references, including database entries, that are cited herein are hereby incorporated by reference to the same extent as if each was specifically and individually indicated to be incorporated by reference.

As used herein, the term “polymorphs” refer to different polymorphic forms of the same compound and includes, but is not limited to, other solid state molecular forms including solvation products and amorphous forms of the same compound. The term “polymorph” refers to any one such form. Different polymorphs of a given compound may differ from each other with respect to one or more physical properties, such as solubility and dissociation, true density, crystal shape, compaction behavior, flow properties, and/or solid state stability. Unstable polymorphs generally convert to the more thermodynamically stable forms at a given temperature after a sufficient period of time. Metastable forms are unstable polymorphs that slowly convert to stable forms. A metastable pharmaceutical solid form can change crystalline structure or solvate/desolvate in response to changes in environmental conditions, processing, or over time. In general, the stable form exhibits the highest melting point and the most chemical stability; however, metastable forms may also have sufficient chemical and physical stability to render them pharmaceutically acceptable. “Chemical stability” refers to stability in chemical properties, such as thermal stability, light stability, and moisture stability.

The recitation of “about” preceding a range of values is intended to modify both endpoints in the range, e.g., “about 83-89° C.” is equivalent to “about 83° C. to about 89° C.”.

As used herein, the recitation of a numerical range for a variable is intended to convey that the invention may be practiced with the variable equal to any of the values within that range. Thus, for a variable which is inherently discrete, the variable can be equal to any integer value within the numerical range, including the end-points of the range. Similarly, for a variable which is inherently continuous, the variable can be equal to any real value within the numerical range, including the end-points of the range. As an example, and without limitation, a variable which is described as having values between 0 and 2 can take the values 0, 1 or 2 if the variable is inherently discrete, and can take the values 0.0, 0.1, 0.01, 0.001, or any other real values ≧0 and ≦2 if the variable is inherently continuous.

As used herein, unless specifically indicated otherwise, the word “or” is used in the inclusive sense of “and/or” and not the exclusive sense of “either/or.”

As used herein, “Free Base” refers to 4-(4-(imidazo[1,2-b]pyridazin-2-ylmethoxy)phenyl)-2,2-dimethyl-5-(pyridin-4-yl)furan-3(2H)-one. As used herein, the phrase “crystalline Free Base or a salt thereof” includes, but is not limited to, crystalline Form 1 and Form 2 of 4-(4-(imidazo[1,2-b]pyridazin-2-ylmethoxy)phenyl)-2,2-dimethyl-5-(pyridin-4-yl)furan-3(2H)-one, amorphous salt of 4-(4-(imidazo[1,2-b]pyridazin-2-ylmethoxy)phenyl)-2,2-dimethyl-5-(pyridin-4-yl)furan-3(2H)-one, and crystalline salt of 4-(4-(imidazo[1,2-b]pyridazin-2-ylmethoxy)phenyl)-2,2-dimethyl-5-(pyridin-4-yl)furan-3(2H)-one.

Abbreviations: XRD is X-ray diffraction, XRPD is X-Ray Powder Diffraction, SCXRD is Single Crystal X-Ray Diffraction, DSC is Differential Scanning Calorimetry, HPLC is High Performance Liquid Chromatography, DMSO is dimethyl sulfoxide, IPA is isopropyl alcohol, FASSIF is Fasted State Simulated Intestinal Fluid, FESSIF is Fed State Simulated Intestinal Fluid, ee is enantiomeric excess, GVS is Gravimetric Vapor Sorption, n-BuOAc is n-butyl acetate, EtOAc is ethyl acetate, EtOH is ethanol, i-PrOAc is iso-propyl acetate, i-PrOH is iso-propyl alcohol, MeCN is acetonitrile, MEK is methyl ethyl ketone, MeOH is methanol, MIBK is methyl isobutyl ketone, n-BuOH is n-butanol, n-PrOH is n-propanol, PTFE is polytetrafluoroethene, RH is relative humidity, RM is reaction mixture, RT is room temperature, TBME is t-butyl methyl ether, t-BuOH is t-butanol, THF is tetrahydrofuran.

Disclosed herein are crystalline forms of 4-(4-(imidazo[1,2-b]pyridazin-2-ylmethoxy)phenyl)-2,2-dimethyl-5-(pyridin-4-yl)furan-3(2H)-one (crystalline forms of the Free Base”). In some embodiments, these crystalline forms are non-solvated crystalline materials. Two types of crystalline forms of the Free Base are disclosed, Form I and Form II.

### 2. 4-(4-(imidazo[1,2-b]pyridazin-2-ylmethoxy)phenyl)-2,2-dimethyl-5-(pyridin-4-yl)furan-3(2H)-one, Crystalline Form 1

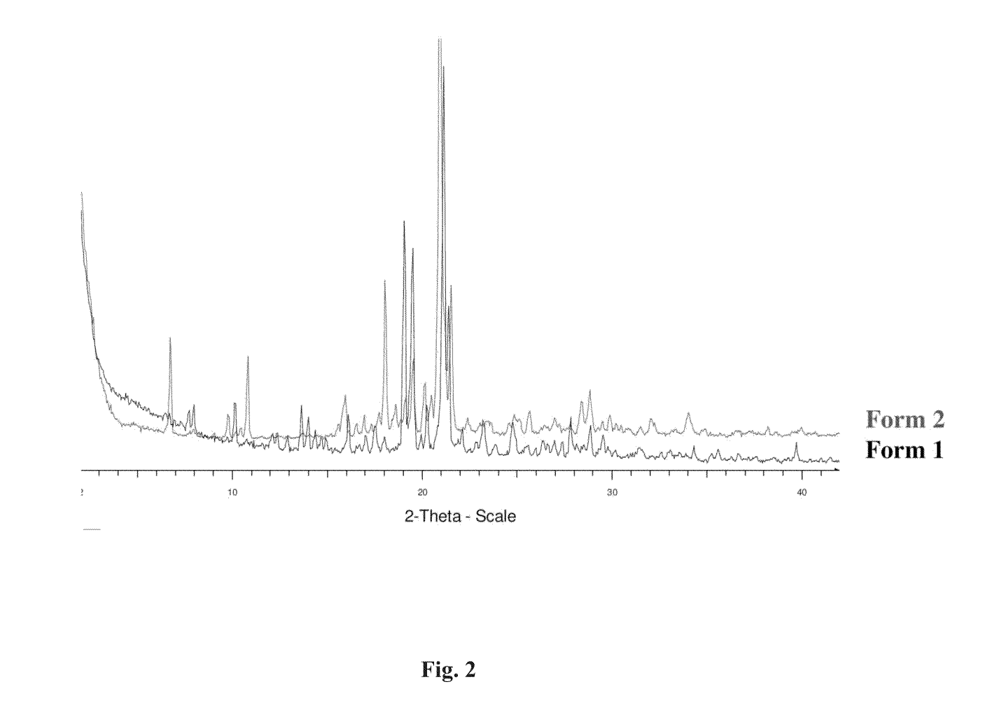

Crystalline Form 1 of 4-(4-(imidazo[1,2-b]pyridazin-2-ylmethoxy)phenyl)-2,2-dimethyl-5-(pyridin-4-yl)furan-3(2H)-one has a peak position at about 7.9, 8.0, 10.2, 13.7, 14.0, 16.2, 17.6, 19.1, 19.3, 21.2, or 21.4 degrees 2-theta in an x-ray powder diffraction pattern obtained using Cu-Kα radiation. In some embodiments, crystalline Form 1 of the Free Base has a peak position at about 19.1, 19.3, 21.2, or 21.4 degrees 2-theta in an x-ray powder diffraction pattern obtained using Cu-Kα radiation. In certain embodiments, crystalline Form 1 of the crystalline form of the Free Base is characterized by the X-ray powder diffractogram substantially as shown in FIG. 2, Form 1. In some embodiments, the Form 1 of the crystalline Free Base can be characterized by respective lattice parameters, a, b, and c of about 12.2 Å, 27.4 Å, and 12.4 Å, respectively, and β of about 96.7° in the monoclinic crystal system P2₁ space group, when measured with Cu-Kα radiation at about 120 K. In some embodiments, the crystalline Form 1 of the Free Base can be also characterized by single crystal structure illustrated by X-Ray diffraction studies. In some embodiments, the crystalline Form 1 of the Free Base can be characterized by one or more of single crystal X-Ray diffraction parameters provided in Table 1.

TABLE 1
Single crystal structure of the Free Base, Form 1.

| Molecular formula | C₂₄H₂₀N₄O₃ | | | | |
|---|---|---|---|---|---|
| Molecular weight | 412.44 | | | | |
| Crystal system | Monoclinic | | | | |
| Space group | P2(1)/c | a | 12.1807(8) Å, | α | 90°, |
| | | b | 27.3606(11) Å, | β | 96.714(4)°, |
| | | c | 12.3608(5) Å, | γ | 90° |

| | |
|---|---|
| V | 4091.2(4) Å³ |
| Z | 8 |
| D_{c} | 1.339 g · cm⁻¹ |
| μ | 0.738 mm⁻¹ |
| Source, λ | Cu—Kα, 1.5418 Å |
| F(000) | 1728 |
| T | 120(1) K |
| Crystal | Colorless prism, 0.08 × 0.04 × 0.02 mm |
| Data truncated to | 0.80 Å |
| θₘₐₓ | 74.16° |
| Completeness | 96.2% |
| Reflections | 17121 |
| Unique reflections | 8011 |
| Rᵢₙₜ | 0.0646 |

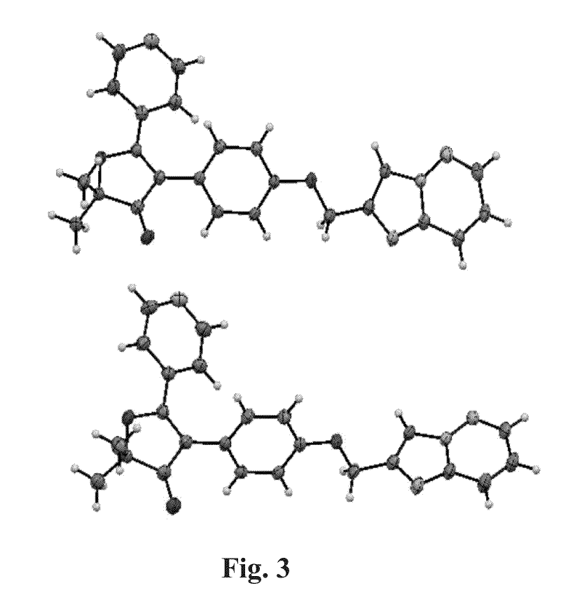

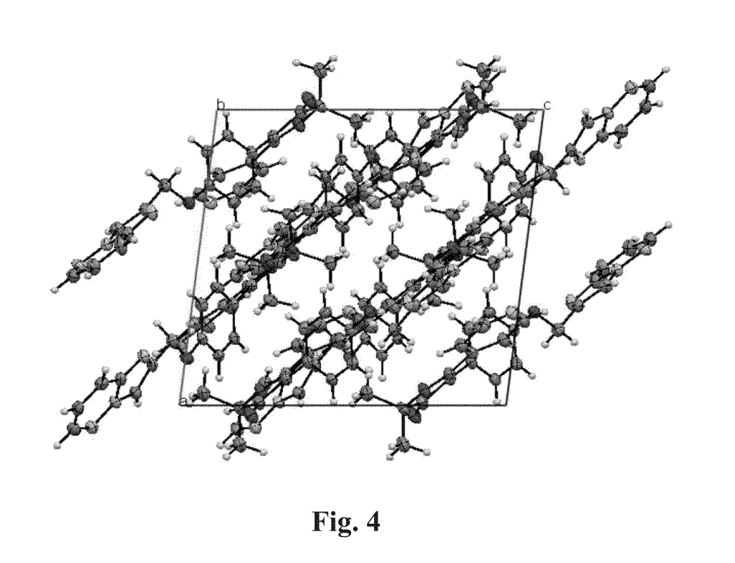

In certain embodiments, the crystalline Form 1 of the Free Base can be characterized as having the molecular confirmation substantially as shown in FIG. 3. In certain embodiments, the crystalline Form 1 of the Free Base can be characterized as having the crystal packing substantially as shown in FIG. 4.

The crystalline Form 1 of the Free Base is isolated in high chemical purity. In some embodiments, the crystalline Form 1 of the Free Base is at least about 90%, 95%, 97%, 98%, or 99% chemically pure. The chemical purity of the crystalline Form 1 can be determined by HPLC or any other methods known in the art.

It was seen discovered that the crystalline Form 1 of the Free Base is highly stable. In some embodiments, the crystalline Form 1 of the Free Base is stable at room temperature under air for at least about 4, 6, 8, 10, 12, or 20 weeks. The crystalline Form 1 of the Free Base is also stable at elevated temperature and/or humidity as well. In certain embodiments, the crystalline Form 1 of the Free Base is stable at 40° C., or 60° C. for at least 4, 6, 8, or 10 weeks. In certain embodiments, the crystalline Form 1 of the Free Base is stable at 60%, 75%, or 96% humidity at 25° C., 40° C., or 60° C. for at least 4, 6, 8, or 10 weeks.

In certain embodiments, the crystalline Form 1 of the Free Base has a melting point from about 180° C. to about 190° C. In some embodiments, the crystalline Form 1 of the Free Base has a melting point from about 184° C. to about 186° C., or about 185° C.

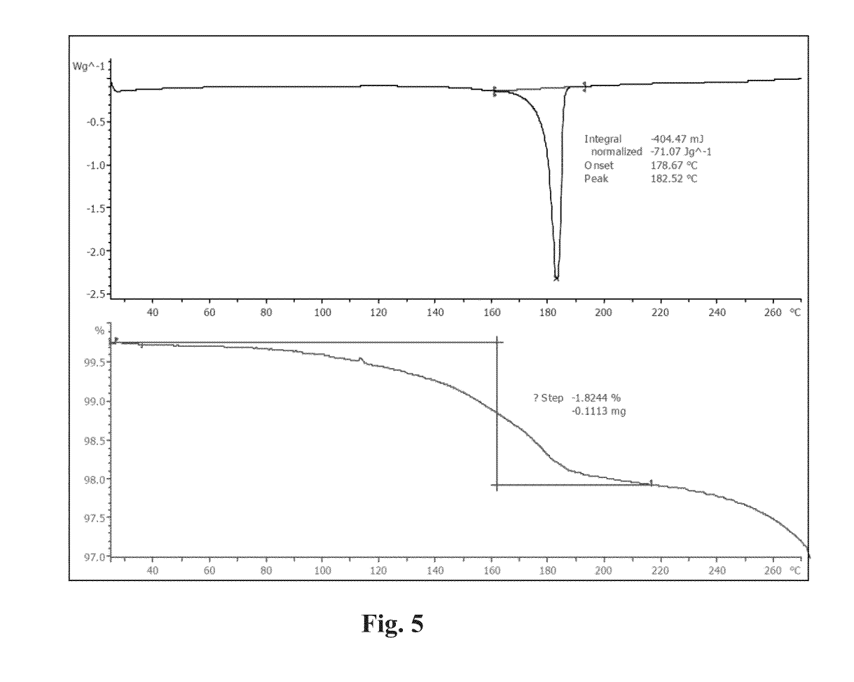

The crystalline Form 1 of the Free Base can also be characterized by a differential scanning calorimetry (DSC) thermogram. In certain embodiments, the DSC thermogram of the crystalline Form 1 of the Free Base has an endothermic onset at about 180-190° C. The endothermic onset can be at about 185-186° C., or about 185° C. The crystalline Form 1 of the Free Base can be characterized by the differential scanning calorimetry (DSC) thermogram substantially as shown in FIG. 5.

The crystalline Form 1 of the Free Base is soluble in deionized water. The thermodynamic aqueous solubility of crystalline Form 1 of the Free Base is about 0.06 mg/ml at pH 7.67.

### 3. 4-(4-(imidazo[1,2-b]pyridazin-2-ylmethoxy)phenyl)-2,2-dimethyl-5-(pyridin-4-yl)furan-3(2H)-one, Crystalline Form 2

The crystalline Form 2 of 4-(4-(imidazo[1,2-b]pyridazin-2-ylmethoxy)phenyl)-2,2-dimethyl-5-(pyridin-4-yl)furan-3(2H)-one has a peak position at about 6.7, 10.8, 15.8, 18.0, 19.4, 20.2, 21.1, 21.5, or 28.8 degrees 2-theta in an x-ray powder diffraction pattern obtained using Cu-Kα radiation. In some embodiments, the crystalline Form 2 of the Free Base has a peak position at about 6.7, 10.8, 18.0, 19.4, 21.1, or 21.5 degrees 2-theta in an x-ray powder diffraction pattern obtained using Cu-Kα radiation. In certain embodiments, the crystalline Form 2 of the Free Base is characterized by the X-ray powder diffractogram substantially as shown in FIG. 2, Form 2. In some embodiments, the crystalline Form 2 of the Free Base can be characterized by respective lattice parameters, a, b, and c of about 11.9 Å, 18.0 Å, and 19.4 Å, respectively, and β of about 102.8° in the monoclinic crystal system P2₁ space group, when measured with Cu-Kα radiation at about 100 K. In some embodiments, the crystalline Form 2 of the Free Base can be also characterized by single crystal structure illustrated by X-Ray diffraction studies. In some embodiments, the crystalline Form 2 of the Free Base can be characterized by one or more single crystal X-Ray diffraction parameters provided in Table 2.

TABLE 2
Single crystal structure of the Free Base, Form 2.

| Molecular formula | C₂₄H₂₀N₄O₃ | | | | |
|---|---|---|---|---|---|
| Molecular weight | 412.44 | | | | |
| Crystal system | Monoclinic | | | | |
| Space group | P2(1)/n | a | 11.8987(2) Å, | α | 90°, |
| | | b | 18.0160(2) Å, | β | 102.771(2)°, |
| | | c | 19.3713(3) Å, | γ | 90° |

| | |
|---|---|
| V | 4049.84(10) Å³ |
| Z | 8 |
| D_{c} | 1.353 g · cm⁻¹ |
| μ | 0.746 mm⁻¹ |
| Source, λ | Cu—Kα, 1.54178 Å |
| F(000) | 1728 |
| T | 100(1) K |
| Crystal | Colorless block, 0.55 × 0.35 × 0.35 mm |
| Data truncated to | 0.80 Å |
| θₘₐₓ | 66.59° |
| Completeness | 99.5% |
| Reflections | 14804 |
| Unique reflections | 7125 |
| Rᵢₙₜ | 0.0277 |

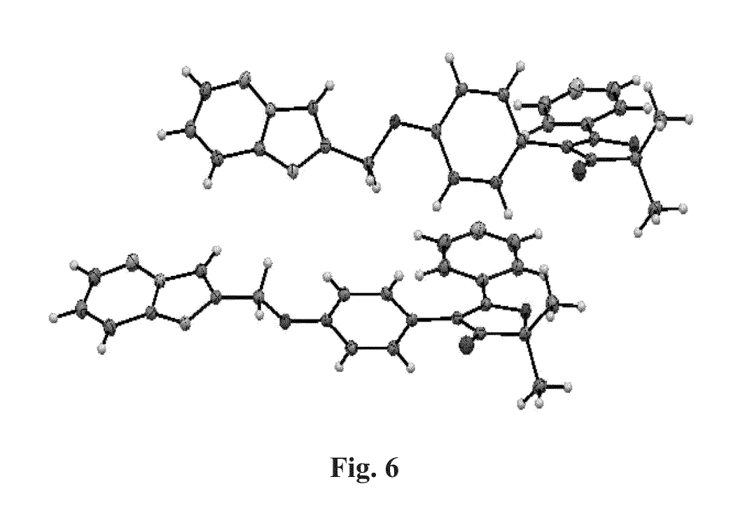

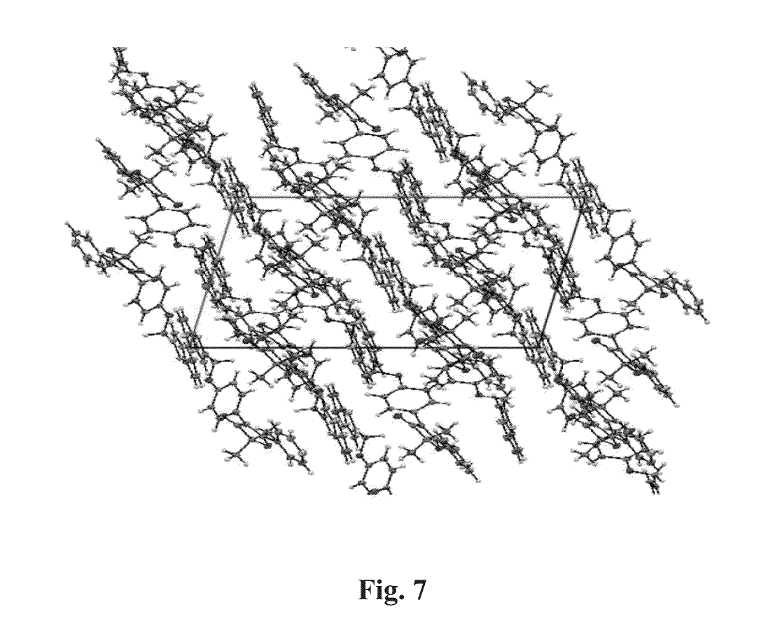

In certain embodiments, the crystalline Form 2 of the Free Base can be characterized as having the molecular confirmation substantially as shown in FIG. 6. In certain embodiments, the crystalline Form 2 of the Free Base can be characterized as having the crystal packing substantially as shown in FIG. 7.

The crystalline Form 2 of the Free Base is isolated in high chemical purity. In some embodiments, the crystalline Form 2 of the Free Base is at least about 90%, 95%, 97%, 98%, or 99% chemically pure. The chemical purity of the crystalline Form 2 of the Free Base can be determined by HPLC or any other methods known in the art.

It is also discovered that the crystalline Form 2 of the Free Base is highly stable. In some embodiments, the crystalline Form 2 of the Free Base is stable at room temperature under air for at least about 4, 6, 8, 10, 12, or 20 weeks. The crystalline Form 2 of the Free Base is also stable at elevated temperature and/or humidity as well. In certain embodiments, the crystalline Form 2 of the Free Base is stable at 40° C., or 60° C. for at least 4, 6, 8, or 10 weeks. In certain embodiments, the crystalline Form 2 of the Free Base is stable at 60%, 75%, or 96% humidity at 25° C., 40° C., or 60° C. for at least 4, 6, 8, or 10 weeks.

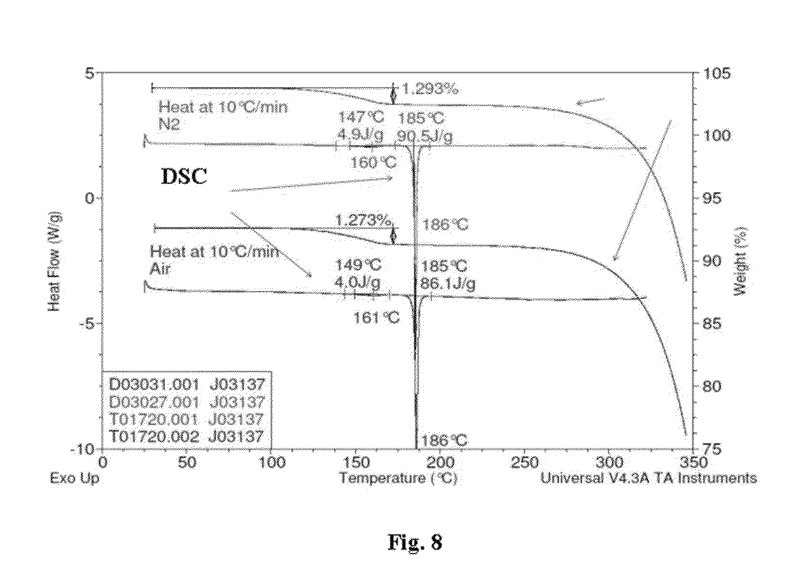

The crystalline Form 2 of the Free Base can also be characterized by a differential scanning calorimetry (DSC) thermogram. In certain embodiment, the crystalline Form 2 of the Free Base has an endothermic onset at about 140-165° C. in a DSC profile. The endothermic onset can be at about 145° C. It is discovered that the crystalline Form 2 of the Free Base can convert to the crystalline Form 1 of the Free Base at about 140-165° C., or about 145° C. upon heating. The crystalline Form 2 of the Free Base can be characterized by the differential scanning calorimetry (DSC) thermogram substantially as shown in FIG. 8, where an endothermic onset at about 140-165° C. indicates that the crystalline Form 2 of the Free Base was converted to the crystalline Form 1 of the Free Base which has an endothermic onset at about 185° C.

The crystalline Form 2 of the Free Base is soluble in deionized water. The thermodynamic aqueous solubility of crystalline Form 2 of the Free Base is about 0.04 mg/ml at pH 7.23.

Synthesis of 4-(4-(imidazo[1,2-b]pyridazin-2-ylmethoxy)phenyl)-2,2-dimethyl-5-(pyridin-4-yl)furan-3(2H)-one is described in U.S. Pat. No. 8,343,973, filed on Dec. 18, 2009, which is incorporated by reference herein.

### 4. 4-(4-(imidazo[1,2-b]pyridazin-2-ylmethoxy)phenyl)-2,2-dimethyl-5-(pyridin-4-yl)furan-3(2H)-one Salts

Also disclosed herein are the salts of 4-(4-(imidazo[1,2-b]pyridazin-2-ylmethoxy)phenyl)-2,2-dimethyl-5-(pyridin-4-yl)furan-3(2H)-one. The salts of 4-(4-(imidazo[1,2-b]pyridazin-2-ylmethoxy)phenyl)-2,2-dimethyl-5-(pyridin-4-yl)furan-3(2H)-one maybe amorphous or crystalline.

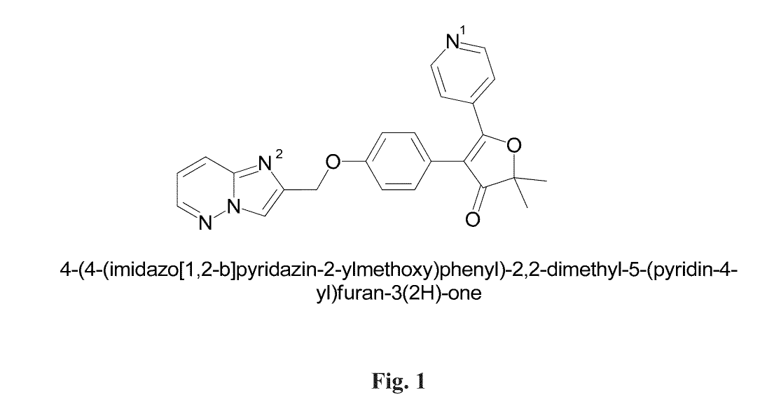

4-(4-(Imidazo[1,2-b]pyridazin-2-ylmethoxy)phenyl)-2,2-dimethyl-5-(pyridin-4-yl)furan-3(2H)-one has two basic nitrogens: N¹ and N² as illustrated in FIG. 1, both of which can form a salt with an acid. In some embodiments, the salt of 4-(4-(Imidazo[1,2-b]pyridazin-2-ylmethoxy)phenyl)-2,2-dimethyl-5-(pyridin-4-yl)furan-3(2H)-one is a mono-salt, i.e., the molar ratio of acid:4-(4-(Imidazo[1,2-b]pyridazin-2-ylmethoxy)phenyl)-2,2-dimethyl-5-(pyridin-4-yl)furan-3(2H)-one in the salt is about 1:1. The mono-salt can be formed by N¹ or N² of 4-(4-(Imidazo[1,2-b]pyridazin-2-ylmethoxy)phenyl)-2,2-dimethyl-5-(pyridin-4-yl)furan-3(2H)-one with an acid. In other embodiments, the salt of 4-(4-(Imidazo[1,2-b]pyridazin-2-ylmethoxy)phenyl)-2,2-dimethyl-5-(pyridin-4-yl)furan-3(2H)-one is a bis-salt, i.e., the molar ratio of acid:4-(4-(Imidazo[1,2-b]pyridazin-2-ylmethoxy)phenyl)-2,2-dimethyl-5-(pyridin-4-yl)furan-3(2H)-one is 2:1. In these embodiments, the bis-salt can also be a mixed salt, wherein two different types of acids are used to form the bis-salt, wherein the overall ratio of the two acids:4-(4-(Imidazo[1,2-b]pyridazin-2-ylmethoxy)phenyl)-2,2-dimethyl-5-(pyridin-4-yl)furan-3(2H)-one in the salt is 2:1.

In some embodiments, the acid used to form the salt is an organic acid. In other embodiments, the acid used to form the salt is an inorganic acid. In still other embodiments, a mixture of an organic acid and an inorganic acid is used to form a bis salt of 4-(4-(Imidazo[1,2-b]pyridazin-2-ylmethoxy)phenyl)-2,2-dimethyl-5-(pyridin-4-yl)furan-3(2H)-one.

In some embodiments, the acid used to form the salt of 4-(4-(Imidazo[1,2-b]pyridazin-2-ylmethoxy)phenyl)-2,2-dimethyl-5-(pyridin-4-yl)furan-3(2H)-one is selected from the group consisting of HCl, HBr, HI, H₂SO₄, H₃PO₄, HNO₃, trifluoroacetic acid, acetic acid, propanoic acid, tosic acid, p-toluene sulphonic acid, benzene sulphonic acid, methanesulphonic acid, oxalic acid, fumaric acid, L-aspartic acid or any other types of amino acids, maleic acid, 1-hydroxy-2-naphthoic acid, malonic acid, L-tartatic acid, citric acid, and L-malic acid. Any other suitable acid known in the art can be used to form the salt with the Free Base. Mono- or bis-salt of 4-(4-(Imidazo[1,2-b]pyridazin-2-ylmethoxy)phenyl)-2,2-dimethyl-5-(pyridin-4-yl)furan-3(2H)-one can be formed with one or more of these acids.

In some embodiments, the salt of 4-(4-(Imidazo[1,2-b]pyridazin-2-ylmethoxy)phenyl)-2,2-dimethyl-5-(pyridin-4-yl)furan-3(2H)-one is a mono-salt formed by 1 equivalent of 4-(4-(Imidazo[1,2-b]pyridazin-2-ylmethoxy)phenyl)-2,2-dimethyl-5-(pyridin-4-yl)furan-3(2H)-one and 1 equivalent of HCl, HBr, HF, HI, H₂SO₄, H₃PO₄, HNO₃, trifluoroacetic acid, acetic acid, propanoic acid, tosic acid, p-toluene sulphonic acid, benzene sulphonic acid, methanesulphonic acid, oxalic acid, fumaric acid, L-aspartic acid or any other types of amino acids, maleic acid, 1-hydroxy-2-naphthoic acid, malonic acid, L-tartatic acid, citric acid, or L-malic acid.

In some embodiments, the salt of 4-(4-(Imidazo[1,2-b]pyridazin-2-ylmethoxy)phenyl)-2,2-dimethyl-5-(pyridin-4-yl)furan-3(2H)-one is a bis-salt. In certain embodiments, the bis-salt is bis-oxalate salt, bis-MsOH salt, bis-HCl salt, bis-HBr, or bis-Tosylate salt of 4-(4-(Imidazo[1,2-b]pyridazin-2-ylmethoxy)phenyl)-2,2-dimethyl-5-(pyridin-4-yl)furan-3(2H)-one.

Various solvents can be used in the formation of the salt. Non-limiting examples of suitable solvents include water, EtOH, MeOH, THF, EtOAc, CH₃CN, DMF, DMSO, propanol, CH₂Cl₂, and toluene.

In some embodiments, the salt of 4-(4-(Imidazo[1,2-b]pyridazin-2-ylmethoxy)phenyl)-2,2-dimethyl-5-(pyridin-4-yl)furan-3(2H)-one may be a hydrate which includes one or more equivalent of the water molecule.

The salt of 4-(4-(Imidazo[1,2-b]pyridazin-2-ylmethoxy)phenyl)-2,2-dimethyl-5-(pyridin-4-yl)furan-3(2H)-one, disclosed herein, may be crystalline and can be characterized by a characteristic XRPD diffraction pattern and/or a well-defined DSC thermogram. In other embodiments, the salts disclosed herein are amorphous.

In some embodiments, chemical purity of a crystalline or amorphous salt is determined by HPLC, e.g., by measuring the area under the peak representing the salt and comparing it to the area of non-solvent peaks. Other methods well-known in the art, e.g., NMR, may also be used to determine the chemical purity of the salt.

The salt 4-(4-(Imidazo[1,2-b]pyridazin-2-ylmethoxy)phenyl)-2,2-dimethyl-5-(pyridin-4-yl)furan-3(2H)-one can be prepared and characterized as described in the Examples. Any other methods known in the art can be used in preparing these salts.

### 5. Pharmaceutical Compositions Comprising a Crystalline Free Base or a Salt Thereof and their Administration

Due to their activity, the crystalline Free Base or a salt thereof is advantageously useful in human medicine. As described above, the crystalline Free Base or a salt thereof is useful for treating CNS disorders, eating disorders, obesity, compulsive gambling, sexual disorders, narcolepsy, sleep disorders, diabetes, metabolic syndrome, schizophrenia, schizo-affective conditions, Huntington's disease, bipolar disorders, dystonic conditions and tardive dyskinesia, or for use in smoking cessation treatment.

The crystalline Free Base or a salt thereof can be administered in amounts that are effective to treat or prevent a neurological disorder (e.g., schizophrenia or Huntington's disease) in a subject in need thereof.

In one embodiment, the treatment of CNS disorders and conditions by the crystalline Free Base or a salt thereof of the disclosure can include Huntington's disease, schizophrenia and schizo-affective conditions, delusional disorders, drug-induced psychoses, panic and obsessive compulsive disorders, post-traumatic stress disorders, age-related cognitive decline, attention deficit/hyperactivity disorder, bipolar disorders, personality disorders of the paranoid type, personality disorders of the schizoid type, psychosis induced by alcohol, amphetamines, phencyclidine, opioids hallucinogens or other drug-induced psychosis, dyskinesia or choreiform conditions including dyskinesia induced by dopamine agonists, dopaminergic therapies, psychosis associated with Parkinson's disease, psychotic symptoms associated with other neurodegenerative disorders including Alzheimer's disease, dystonic conditions such as idiopathic dystonia, drug-induced dystonia, torsion dystonia, and tardive dyskinesia, mood disorders including major depressive episodes, post-stroke depression, minor depressive disorder, premenstrual dysphoric disorder, dementia including, but not limited to, multi-infarct dementia, AIDS-related dementia, and neurodegenerative dementia.

In another embodiment, the crystalline Free Base or a salt thereof of the disclosure may be used for the treatment of eating disorders, obesity, compulsive gambling, sexual disorders, narcolepsy, sleep disorders as well as in smoking cessation treatment.

In a further embodiment, the crystalline Free Base or a salt thereof of the disclosure may be used for the treatment of obesity, schizophrenia, schizo-affective conditions, Huntington's disease, dystonic conditions and tardive dyskinesia.

In another embodiment, the crystalline Free Base or a salt thereof of the disclosure may be used for the treatment of schizophrenia, schizo-affective conditions, Huntington's disease and obesity.

In a further embodiment, the crystalline Free Base or a salt thereof of the disclosure may be used for the treatment of schizophrenia and/or schizo-affective conditions.

In an additional embodiment, the crystalline Free Base or a salt thereof of the disclosure may be used for the treatment of Huntington's disease.

In another embodiment, the crystalline Free Base or a salt thereof of the disclosure may be used for the treatment of obesity and metabolic syndrome.

The crystalline Free Base or a salt thereof of the disclosure may also be used in mammals and humans in conjunction with conventional antipsychotic medications including, but not limited to, Clozapine, Olanzapine, Risperidone, Ziprasidone, Haloperidol, Aripiprazole, Sertindole and Quetiapine. The combination of a compound of Formula (I) with a subtherapeutic dose of an aforementioned conventional antipsychotic medication may afford certain treatment advantages including improved side effect profiles and lower dosing requirements.

When administered to a subject, a crystalline Free Base or a salt thereof can be administered as a component of a composition that comprises a physiologically acceptable carrier or vehicle. The present compositions, which comprise a crystalline Free Base or a salt thereof, can be administered orally. A crystalline Free Base or a salt thereof can also be administered by any other convenient route, for example, by infusion or bolus injection, by absorption through epithelial or mucocutaneous linings (e.g., oral, rectal, or intestinal mucosa) and can be administered as the sole active pharmaceutical ingredient or together with another biologically active agent. Administration of a crystalline Free Base or a salt thereof can be systemic or local. Various delivery systems are known, e.g., encapsulation in liposomes, microparticles, microcapsules and capsules.

Exemplary methods of administration of a crystalline Free Base or a salt thereof include, but are not limited to, intradermal, intramuscular, intraperitoneal, intravenous, subcutaneous, intranasal, epidural, oral, sublingual, intracerebral, intravaginal, transdermal, rectal, by inhalation, or topical, specifically to the ears, nose, eyes, or skin. In some instances, administration results in the release of crystalline Free Base or a salt thereof into the bloodstream.

In one embodiment, the crystalline Free Base or a salt thereof is administered orally. In other embodiments, it can be desirable to administer the crystalline Free Base or a salt thereof locally. This can be achieved, for example, and not by way of limitation, by local infusion during surgery, topical application, e.g., in conjunction with a wound dressing after surgery, by injection, by means of a catheter, by means of a suppository or enema, or by means of an implant, said implant being of a porous, non-porous, or gelatinous material, including membranes, such as sialastic membranes, or fibers.

In certain embodiments, it can be desirable to introduce the crystalline Free Base or a salt thereof into the central nervous system or gastrointestinal tract by any suitable route, including intraventricular, intrathecal, and epidural injection, and enema. Intraventricular injection can be facilitated by an intraventricular catheter, for example, attached to a reservoir, such as an Ommaya reservoir.

Pulmonary administration can also be employed, e.g., by use of an inhaler of nebulizer, and formulation with an aerosolizing agent, or via perfusion in a fluorocarbon or a synthetic pulmonary surfactant. In certain embodiments, the crystalline Free Base or a salt thereof can be formulated as a suppository, with traditional binders and excipients such as triglycerides.

In another embodiment, the crystalline Free Base or a salt thereof can be delivered in a vesicle, specifically a liposome (see Langer, Science 249:1527-1533 (1990) and Liposomes in Therapy of Infectious Disease and Cancer 317-327 and 353-365, Lopez-Berestein and Fidler (eds.), Liss, New York (1989)).

In yet another embodiment, the crystalline Free Base or a salt thereof can be delivered in a controlled-release system or sustained release system (see, e.g., Goodson, in Medical Applications of Controlled Release, supra, vol. 2, pp. 115-138 (1984)). Other controlled or sustained release systems discussed in the review by Langer, Science 249:1527-1533 (1990) can be used. In one embodiment, a pump can be used (Langer, Science 249:1527-1533 (1990); Sefton, CRC Crit. Ref. Biomed. Eng. 14:201 (1987); Buchwald et al., Surgery 88:507 (1980); and Saudek et al., N. Engl. J Med. 321:574 (1989)). In another embodiment, polymeric materials can be used (see Medical Applications of Controlled Release (Langer and Wise eds., 1974); Controlled Drug Bioavailability, Drug Product Design and Performance (Smolen and Ball eds., 1984); Ranger and Peppas, J. Macromol. Sci. Rev. Macromol. Chem. 2:61 (1983); Levy et al., Science 228:190 (1935); During et al., Ann. Neural. 25:351 (1989); and Howard et al., J. Neurosurg. 71:105 (1989)).

In yet another embodiment, a controlled or sustained release system can be placed in proximity of a target of a crystalline Free Base or a salt thereof, e.g., the spinal column, brain, skin, lung, or gastrointestinal tract, thus requiring only a fraction of the systemic dose.

In one embodiment, the crystalline Free Base or a salt thereof can be administered by introduction into the central nervous system of the subject, e.g., into the cerebrospinal fluid of the subject. The formulations for administration will commonly comprise a solution of a crystalline Free Base or a salt thereof dissolved in a pharmaceutically acceptable carrier. In certain aspects, a crystalline Free Base or a salt thereof is introduced intrathecally, e.g., into a cerebral ventricle, the lumbar area, or the cisterna magna. In another aspect, a crystalline Free Base or a salt thereof is introduced intraocularly, to thereby contact retinal ganglion cells.

In one embodiment, the pharmaceutical composition comprising a crystalline Free Base or a salt thereof is administered into a subject intrathecally. As used herein, the term “intrathecal administration” is intended to include delivering a pharmaceutical composition comprising a crystalline Free Base or a salt thereof directly into the cerebrospinal fluid of a subject, by techniques including lateral cerebroventricular injection through a burrhole or cisternal or lumbar puncture or the like (described in Lazorthes et al., Advances in Drug Delivery Systems and Applications in Neurosurgery, 143-192 and Omaya et al., Cancer Drug Delivery, 1: 169-179). The term “lumbar region” is intended to include the area between the third and fourth lumbar (lower back) vertebrae. The term “cisterna magna” is intended to include the area where the skull ends and the spinal cord begins at the back of the head. The term “cerebral ventricle” is intended to include the cavities in the brain that are continuous with the central canal of the spinal cord. Administration of a crystalline Free Base or a salt thereof to any of the above mentioned sites can be achieved by direct injection of the pharmaceutical composition comprising the crystalline Free Base or a salt thereof or by the use of infusion pumps. For injection, the pharmaceutical compositions can be formulated in liquid solutions, preferably in physiologically compatible buffers such as Hank's solution or Ringer's solution. In addition, the pharmaceutical compositions may be formulated in solid form and re-dissolved or suspended immediately prior to use. Lyophilized forms are also included. The injection can be, for example, in the form of a bolus injection or continuous infusion (e.g., using infusion pumps) of pharmaceutical composition.

In one embodiment, the pharmaceutical composition comprising a crystalline Free Base or a salt thereof is administered by lateral cerebro ventricular injection into the brain of a subject. The injection can be made, for example, through a burr hole made in the subject's skull. In another embodiment, the encapsulated therapeutic agent is administered through a surgically inserted shunt into the cerebral ventricle of a subject. For example, the injection can be made into the lateral ventricles, which are larger, even though injection into the third and fourth smaller ventricles can also be made.

In yet another embodiment, the pharmaceutical composition comprising a crystalline Free Base or a salt thereof is administered by injection into the cisterna magna, or lumbar area of a subject.

The present compositions optionally comprise a suitable amount of one or more pharmaceutically acceptable excipients so as to provide the form for proper administration to the subject.

Such pharmaceutical excipients can be liquids, such as water and oils, including those of petroleum, animal, vegetable, or synthetic origin, such as peanut oil, soybean oil, mineral oil, sesame oil and the like. The pharmaceutical excipients can be saline, gum acacia, gelatin, starch paste, talc, keratin, colloidal silica, urea and the like. In addition, auxiliary, stabilizing, thickening, lubricating, and coloring agents can be used. In one embodiment, the pharmaceutically acceptable excipients are sterile when administered to a subject. Water can be a useful excipient when the crystalline Free Base or a salt thereof is administered intravenously. Saline solutions and aqueous dextrose and glycerol solutions can also be employed as liquid excipients, specifically for injectable solutions. Suitable pharmaceutical excipients also include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The present compositions, if desired, can also contain minor amounts of wetting or emulsifying agents, or pH buffering agents.

The present compositions can take the form of solutions, suspensions, emulsion, tablets, pills, pellets, capsules, capsules containing liquids, powders, sustained release formulations, suppositories, emulsions, aerosols, sprays, suspensions, or any other form suitable for use. In one embodiment, the composition is in the form of a capsule (see, e.g., U.S. Pat. No. 5,698,155). Other examples of suitable pharmaceutical excipients are described in Remington's Pharmaceutical Sciences 1447-1676 (Alfonso R. Gennaro eds., 19th ed. 1995).

In one embodiment, a crystalline Free Base or a salt thereof is formulated in accordance with routine procedures as a composition adapted for oral administration to human beings. Compositions for oral delivery can be in the form of tablets, lozenges, aqueous or oily suspensions, granules, powders, emulsions, capsules, syrups, or elixirs for example. Orally administered compositions can contain one or more agents, for example, sweetening agents such as fructose, aspartame or saccharin; flavoring agents such as peppermint, oil of wintergreen, or cherry; coloring agents; and preserving agents, to provide a pharmaceutically palatable preparation. Moreover, where in tablet or pill form, the compositions can be coated to delay disintegration and absorption in the gastrointestinal tract thereby providing a sustained action over an extended period of time. Selectively permeable membranes surrounding an osmotically active driving a crystalline Free Base or a salt thereof is also suitable for orally administered compositions. In these latter platforms, fluid from the environment surrounding the capsule is imbibed by the driving compound, which swells to displace the agent or agent composition through an aperture. These delivery platforms can provide an essentially zero order delivery profile as opposed to the spiked profiles of immediate release formulations. A time delay material such as glycerol monostearate or glycerol stearate can also be useful. Oral compositions can include standard excipients such as mannitol, lactose, starch, magnesium stearate, sodium saccharin, cellulose, and magnesium carbonate. In one embodiment, the excipients are of pharmaceutical grade.

Pharmaceutical preparations for oral use can be obtained through combination of a crystalline Free Base or a salt thereof with a solid excipient, optionally grinding a resulting mixture, and processing the mixture of granules, after adding suitable additional compounds, if desired, to obtain tablets or dragee cores. Suitable solid excipients in addition to those previously mentioned are carbohydrate or protein fillers that include, but are not limited to, sugars, including lactose, sucrose, mannitol, or sorbitol; starch from corn, wheat, rice, potato, or other plants; cellulose such as methyl cellulose, hydroxypropylmethyl-cellulose or sodium carboxymethylcellulose; and gums including arabic and tragacanth; as well as proteins such as gelatin and collagen. If desired, disintegrating or solubilizing agents may be added, such as the cross-linked polyvinyl pyrrolidone, agar, alginic acid, or a salt thereof, such as sodium alginate.

Capsules for oral use include hard gelatin capsules in which the active ingredient is mixed with a solid diluent, and soft gelatin capsules wherein the active ingredients is mixed with water or an oil such as peanut oil, liquid paraffin or olive oil.

Dragee cores are provided with suitable coatings. For this purpose, concentrated sugar solutions may be used, which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of active compound doses.

In another embodiment, the crystalline Free Base or a salt thereof can be formulated for intravenous administration. Typically, compositions for intravenous administration comprise sterile isotonic aqueous buffer. Where necessary, the compositions can also include a solubilizing agent. Compositions for intravenous administration can optionally include a local anesthetic such as lignocaine to lessen pain at the site of the injection. Generally, the ingredients are supplied either separately or mixed together in unit dosage form, for example, as a dry lyophilized powder or water-free concentrate in a hermetically sealed container such as an ampule or sachette indicating the quantity of active agent. Where the crystalline Free Base or a salt thereof is to be administered by infusion, they can be dispensed, for example, with an infusion bottle containing sterile pharmaceutical grade water or saline. Where the crystalline Free Base or a salt thereof is administered by injection, an ampule of sterile water for injection or saline can be provided so that the ingredients can be mixed prior to administration.

The crystalline Free Base or a salt thereof can be administered by controlled-release or sustained release means or by delivery devices that are well known to those of ordinary skill in the art. Examples include, but are not limited to, those described in U.S. Pat. Nos. 3,845,770; 3,916,899; 3,536,809; 3,598,123; 4,008,719; 5,674,533; 5,059,595; 5,591,767; 5,120,548; 5,073,543; 5,639,476; 5,354,556; and 5,733,556, each of which is incorporated herein by reference in its entirety. Such dosage forms can be useful for providing controlled or sustained release of one or more active ingredients using, for example, hydroxypropylmethyl cellulose, other polymer matrices, gels, permeable membranes, osmotic systems, multilayer coatings, microparticles, liposomes, microspheres, or a combination thereof to provide the desired release profile in varying proportions. Suitable controlled or sustained release formulations known to those skilled in the art, including those described herein, can be readily selected for use with the active ingredients of the invention. The invention thus encompasses single unit dosage forms suitable for oral administration such as, but not limited to, tablets, capsules, gelcaps, and caplets that are adapted for controlled or sustained release.

In one embodiment, a controlled or sustained release composition comprises a minimal amount of a crystalline Free Base or a salt thereof to treat CNS disorders, eating disorders, obesity, compulsive gambling, sexual disorders, narcolepsy, sleep disorders, diabetes, metabolic syndrome, schizophrenia, schizo-affective conditions, Huntington's disease, bipolar disorders, dystonic conditions and tardive dyskinesia, or for use in smoking cessation treatment in a patient over a period of time. Advantages of controlled or sustained release compositions include extended activity of the drug, reduced dosage frequency, and increased subject compliance. In addition, controlled or sustained release compositions can favorably affect the time of onset of action or other characteristics, such as blood levels of a crystalline Free Base or a salt thereof, and can thus reduce the occurrence of adverse side effects.

Controlled or sustained release compositions can initially release an amount of a crystalline Free Base or a salt thereof that promptly produces the desired therapeutic or prophylactic effect, and gradually and continually release other amounts of the crystalline Free Base or a salt thereof to maintain this level of therapeutic or prophylactic effect over an extended period of time. To maintain a constant level of the crystalline Free Base or a salt thereof in the body, the crystalline Free Base or a salt thereof can be released from the dosage form at a rate that will replace the amount of the crystalline Free Base or a salt thereof being metabolized and excreted from the body. Controlled or sustained release of an active ingredient can be stimulated by various conditions, including but not limited to, changes in pH, changes in temperature, concentration or availability of enzymes, concentration or availability of water, or other physiological conditions or compounds.

Oil suspensions can be formulated by suspending a crystalline Free Base or a salt thereof in a vegetable oil, such as arachis oil, olive oil, sesame oil or coconut oil, or in a mineral oil such as liquid paraffin; or a mixture of these. The oil suspensions can contain a thickening agent, such as beeswax, hard paraffin or cetyl alcohol. Sweetening agents can be added to provide a palatable oral preparation, such as glycerol, sorbitol or sucrose. These formulations can be preserved by the addition of an antioxidant such as ascorbic acid. As an example of an injectable oil vehicle, see Minto, J. Pharmacol. Exp. Ther. 281:93-102 (1997). The pharmaceutical formulations of the crystalline Free Base or a salt thereof can also be in the form of oil-in-water emulsions. The oily phase can be a vegetable oil or a mineral oil, described above, or a mixture of these. Suitable emulsifying agents include naturally-occurring gums, such as gum acacia and gum tragacanth, naturally occurring phosphatides, such as soybean lecithin, esters or partial esters derived from fatty acids and hexitol anhydrides, such as sorbitan mono-oleate, and condensation products of these partial esters with ethylene oxide, such as polyoxyethylene sorbitan mono-oleate. The emulsion can also contain sweetening agents and flavoring agents, as in the formulation of syrups and elixirs. Such formulations can also contain a demulcent, a preservative, or a coloring agent.

In addition to the formulations described previously, a crystalline Free Base or a salt thereof may also be formulated as a depot preparation. Such long acting formulations may be administered by implantation or transcutaneous delivery (e.g., subcutaneously or intramuscularly), intramuscular injection or a transdermal patch. Thus, for example, the crystalline Free Base or a salt thereof may be formulated with suitable polymeric or hydrophobic materials (e.g., as an emulsion in an acceptable oil) or ion exchange resins, or as sparingly soluble derivatives, for example, as a sparingly soluble salt.

For administration by inhalation, a crystalline Free Base or a salt thereof can be conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges for use in an inhaler or insufflator may be formulated containing a powder mix of the crystalline Free Base or a salt thereof and a suitable powder base such as lactose or starch.

The amount of a crystalline Free Base or a salt thereof that is effective in the treatment or prevention of a CNS disorder can be determined by standard clinical techniques. In addition, in vitro or in vivo assays can optionally be employed to help identify optimal dosage ranges. The precise dose to be employed can also depend on the route of administration, and the seriousness of the condition being treated and can be decided according to the judgment of the practitioner and each subject's circumstances in view of, e.g., published clinical studies. Suitable effective dosage amounts, however, range from about 10 micrograms to about 5 grams about every 4 hours, although they are typically about 500 mg or less per every 4 hours. In one embodiment, the effective dosage is about 0.01 mg, 0.5 mg, about 1 mg, about 50 mg, about 100 mg, about 200 mg, about 300 mg, about 400 mg, about 500 mg, about 600 mg, about 700 mg, about 800 mg, about 900 mg, about 1 g, about 1.2 g, about 1.4 g, about 1.6 g, about 1.8 g, about 2.0 g, about 2.2 g, about 2.4 g, about 2.6 g, about 2.8 g, about 3.0 g, about 3.2 g, about 3.4 g, about 3.6 g, about 3.8 g, about 4.0 g, about 4.2 g, about 4.4 g, about 4.6 g, about 4.8 g, and about 5.0 g, every 4 hours. Equivalent dosages can be administered over various time periods including, but not limited to, about every 2 hours, about every 6 hours, about every 8 hours, about every 12 hours, about every 24 hours, about every 36 hours, about every 48 hours, about every 72 hours, about every week, about every two weeks, about every three weeks, about every month, and about every two months. The effective dosage amounts described herein refer to total amounts administered; that is, if more than one salt of the Free Base, or the Free Base and a salt thereof are administered, the effective dosage amounts correspond to the total amount administered.

Compositions of the crystalline Free Base or a salt thereof can be prepared according to conventional mixing, granulating or coating methods, respectively, and the present compositions can contain, in one embodiment, from about 0.1% to about 99%; and in another embodiment from about 1% to about 70% of the crystalline Free Base or a salt thereof by weight or by volume.

The dosage regimen utilizing the crystalline Free Base or a salt thereof can be selected in accordance with a variety of factors including type, species, age, weight, sex and medical condition of the subject; the severity of the condition to be treated; the route of administration; the renal or hepatic function of the subject; and the specific the crystalline Free Base or a salt thereof employed. A person skilled in the art can readily determine the effective amount of the drug useful for treating or preventing the Alzheimer's disease.

A crystalline Free Base or a salt thereof can be administered in a single daily dose, or the total daily dosage can be administered in divided doses of two, three or four times daily. Furthermore, a crystalline Free Base or a salt thereof can be administered in intranasal form via topical use of suitable intranasal vehicles, or via transdermal routes, using those forms of transdermal skin patches well known to those of ordinary skill in that art. To be administered in the form of a transdermal delivery system, the dosage administration can be continuous rather than intermittent throughout the dosage regimen. Other illustrative topical preparations include creams, ointments, lotions, aerosol sprays and gels, wherein the concentration of a crystalline Free Base or a salt thereof ranges from about 0.1% to about 15%, w/w or w/v.

The crystalline Free Base or a salt thereof can be assayed in vitro or in vivo for the desired therapeutic or prophylactic activity prior to use in humans. Animal model systems can be used to demonstrate safety and efficacy.

The present methods for treating or preventing CNS disorders (e.g., schizophrenia, schizo-affective conditions, Huntington's disease) in a subject in need thereof can further comprise administering another prophylactic or therapeutic agent to the subject being administered a crystalline Free Base or a salt thereof. In one embodiment, the other prophylactic or therapeutic agent is administered in an effective amount. The other prophylactic or therapeutic agent includes, but is not limited to, an anti-inflammatory agent, an anti-renal failure agent, an anti-diabetic agent, and anti-cardiovascular disease agent, an antiemetic agent, a hematopoietic colony stimulating factor, an anxiolytic agent, and an analgesic agent.

In one embodiment, the other prophylactic or therapeutic agent is an agent useful for reducing any potential side effect of a crystalline Free Base or a salt thereof. Such potential side effects include, but are not limited to, nausea, vomiting, headache, low white blood cell count, low red blood cell count, low platelet count, headache, fever, lethargy, a muscle ache, general pain, bone pain, pain at an injection site, diarrhea, neuropathy, pruritis, a mouth sore, alopecia, anxiety or depression. In one embodiment, the crystalline Free Base or a salt thereof can be administered prior to, concurrently with, or after an anti-inflammatory agent, or on the same day, or within 1 hour, 2 hours, 12 hours, 24 hours, 48 hours or 72 hours of each other.

Effective amounts of the other prophylactic or therapeutic agents are well known to those skilled in the art. However, it is well within the skilled artisan's purview to determine the other prophylactic or therapeutic agent's optimal effective amount range. In one embodiment of the invention, where another prophylactic or therapeutic agent is administered to a subject, the effective amount of the crystalline Free Base or a salt thereof is less than its effective amount would be where the other prophylactic or therapeutic agent is not administered. In this case, without being bound by theory, it is believed that the crystalline Free Base or a salt thereof and the other prophylactic or therapeutic agent act synergistically to treat or prevent a CNS disorder.

### 5. Kits

The invention provides kits that can simplify the administration of a crystalline Free Base or a salt thereof to a subject. A typical kit of the invention comprises a unit dosage form of a crystalline Free Base or a salt thereof.

In one embodiment, the unit dosage form is a container, which can be sterile, containing an effective amount of a crystalline Free Base or a salt thereof and a physiologically acceptable carrier or vehicle. The kit can further comprise a label or printed instructions instructing the use of the crystalline Free Base or a salt thereof to treat or prevent a CNS disorder such as Huntington's disease and schizophrenia. The kit can also further comprise a unit dosage form of another prophylactic or therapeutic agent, for example, a container containing an effective amount of the other prophylactic or therapeutic agent. In one embodiment, the kit comprises a container containing an effective amount for treating Huntington's disease and an effective amount of another prophylactic or therapeutic agent. In one embodiment, the kit comprises a container containing an effective amount for treating schizophrenia and an effective amount of another prophylactic or therapeutic agent. Examples of other prophylactic or therapeutic agents include, but are not limited to, those listed above.

### EXAMPLES

This invention is further illustrated by the following examples, which should not be construed as limiting. Those skilled in the art will recognize, or be able to ascertain, using no more than routine experimentation, numerous equivalents to the specific substances and procedures described herein. Such equivalents are intended to be encompassed in the scope of the claims that follow the examples below.

### X-Ray Powder Diffraction (XRPD) Methods

Some X-Ray Powder Diffraction patterns were collected on a Bruker AXS C2 GADDS diffractometer using Cu-Kα radiation (40 kV, 40 mA), automated XYZ stage, laser video microscope for auto-sample positioning and a HiStar 2-dimensional area detector. X-ray optics consists of a single Gael multilayer mirror coupled with a pinhole collimator of 0.3 mm.

The beam divergence, i.e., the effective size of the X-ray beam on the sample, was approximately 4 mm. A θ-θ continuous scan mode was employed with a sample-detector distance of 20 cm which gives an effective 2θ range of 3.2°-29.7°. Typically the sample would be exposed to the X-ray beam for 120 seconds. The software used for data collection was GADDS for WNT and the data were analyzed and presented using Diffrac Plus EVA.

Samples run under ambient conditions were prepared as flat plate specimens using powder as received without grinding. Approximately 1-2 mg of the sample was lightly pressed on a glass slide to obtain a flat surface.

Some X-Ray Powder Diffraction patterns were collected on a Bruker D8 diffractometer using Cu-Kα radiation (40 kV, 40 mA), θ-2θ goniometer, and divergence of V4 and receiving slits, a Ge monochromator and a Lynxeye detector. The software used for data collection was Diffrac Plus XRD Commander and the data were analyzed and presented using Diffrac Plus EVA. Samples were run under ambient conditions as flat plate specimens using powder as received. Approximately 10 mg of the sample was gently packed into a cavity cut into polished, zero background (510) silicon wafer. The sample was rotated in its own plane during analysis. The details of the data collection are: Angular range: 2 to 42° 2θ; Step size: 0.05° 2θ; and Collection time: 0.5 s/step.

### Variable Temperature XRPD (VT-XRPD)

Samples run under non-ambient conditions were mounted on a silicon wafer with heat-conducting compound. The sample was then heated to the appropriate temperature at 20° C./min and subsequently held isothermally for 1 minute before data collection was initiated.

### Differential Scanning Calorimetry (DSC) Methods

DSC data were collected on a TA Instruments Q2000 equipped with a 50 position autosampler. The calibration for thermal capacity was carried out using sapphire and the calibration for energy and temperature was carried out using certified indium. Typically 0.5-3 mg of each sample, in a pin-holed aluminum pan, was heated at 10° C./min from 25° C. to 300° C. A purge of dry nitrogen at 50 ml/min was maintained over the sample. Modulated temperature DSC was carried out using an underlying heating rate of 2° C./min and temperature modulation parameters of ±1.272° C. (amplitude) every 60 seconds (period). The instrument control software was Advantage for Q Series and Thermal Advantage and the data were analyzed using Universal Analysis.

### Single Crystal X-Ray Diffraction (SCXRD) Methods

Data were collected on an Oxford Diffraction Supernova Dual Source, Cu at Zero, Atlas CCD diffractometer equipped with an Oxford Cryosystems Cobra cooling device. The data was collected using CuKα radiation. Structures were typically solved using either the SHELXS or SHELXD programs and refined with the SHELXL program as part of the Bruker AXS SHELXTL suite. Unless otherwise stated, hydrogen atoms attached to carbon were placed geometrically and allowed to refine with a riding isotropic displacement parameter. Hydrogen atoms attached to a heteroatom were located in a difference Fourier synthesis and were allowed to refine freely with an isotropic displacement parameter.

### Thermo-Gravimetric Analysis (TGA)

TGA data were collected on a TA Instruments Q500 TGA, equipped with a 16 position autosampler. The instrument was temperature calibrated using certified Alumel and Nickel. Typically 5-10 mg of each sample was loaded onto a pre-tared aluminum DSC pan and heated at 10° C./min from ambient temperature to 350° C. A nitrogen purge at 60 ml/min was maintained over the sample. The instrument control software was Advantage for Q Series and Thermal Advantage and the data were analyzed using Universal Analysis.

### Chemical Purity Determination by High Performance Liquid Chromatography (HPLC)

Purity analysis was performed on an Agilent HP1100 series system equipped with a diode array detector and using ChemStation software using the method detailed in Table 3. Purity was determined by area of the peak.

TABLE 3
HPLC method parameters for chemical purity determinations.

| Sample Preparation | 0.5 mg/ml in acetonitrile:water 1:1 | | |
|---|---|---|---|
| Column | Supelco Ascentis Express C18, | | |
| | 100 × 4.6 mm, 2.7 μm | | |
| Column Temperature (° C.) | 25 | | |
| Injection (μl) | 5 | | |
| Detection: Wavelength, | 255, 90 nm | | |
| Bandwidth (nm) | | | |
| Flow Rate (ml/min) | 2.0 | | |
| Phase A | 0.1% TFA in water | | |
| Phase B | 0.085% TFA in acetonitrile | | |
| Timetable | Time (min) | % Phase A | % Phase B |
| | 0 | 95 | 5 |
| | 6 | 5 | 95 |
| | 6.2 | 95 | 5 |
| | 8 | 95 | 5 |

### Polarized Light Microscopy (PLM)

Samples were studied on a Leica LM/DM polarized light microscope with a digital video camera for image capture. A small amount of each sample was placed on a glass slide, mounted in immersion oil and covered with a glass slip, the individual particles being separated as well as possible. The sample was viewed with appropriate magnification and partially polarized light, coupled to a λ false-color filter.

### Hot Stage Microscopy (HSM)

Hot Stage Microscopy was carried out using a Leica LM/DM polarized light microscope combined with a Mettler-Toledo MTFP82HT hot-stage and a digital video camera for image capture. A small amount of each sample was placed onto a glass slide with individual particles separated as well as possible. The sample was viewed with appropriate magnification and partially polarized light, coupled to a λ false-color filter, whilst being heated from ambient temperature typically at 10° C./min.

### Gravimetric Vapour Sorption (GVS)

Sorption isotherms were obtained using a SMS DVS Intrinsic moisture sorption analyzer, controlled by DVS Intrinsic Control software v1.0.0.30. The sample temperature was maintained at 25° C. by the instrument controls. The humidity was controlled by mixing streams of dry and wet nitrogen, with a total flow rate of 200 ml/min. The relative humidity was measured by a calibrated Rotronic probe (dynamic range of 1.0-100% RH), located near the sample. The weight change, (mass relaxation) of the sample as a function of % RH was constantly monitored by the microbalance (accuracy ±0.005 mg).

Typically 5-30 mg of sample was placed in a tared mesh stainless steel basket under ambient conditions. The sample was loaded and unloaded at 40% relative humidity (RH) and 25° C. (typical room conditions). A moisture sorption isotherm was performed as outlined below in Table 4 (2 scans giving 1 complete cycle). The standard isotherm was performed at 25° C. at 10% RH intervals over a 0-90% RH range. Data analysis was undertaken in Microsoft Excel using DVS Analysis Suite. The sample was recovered after completion of the isotherm and re-analyzed by XRPD.

TABLE 4
Method parameters for SMS DVS Intrinsic experiments.

| Parameters | Values |
|---|---|
| Adsorption - Scan 1 | 40-90 |
| Desorption/Adsorption - Scan 2 | 90-0, 0-40 |
| Intervals (% RH) | 10 |
| Number of Scans | 4 |
| Flow rate (ml/min) | 200 |
| Temperature (° C.) | 25 |
| Stability (° C./min) | 0.2 |
| Sorption Time (hours) | 6 hour time out |

### Thermodynamic Aqueous Solubility

Aqueous solubility was determined by suspending sufficient compound in water or aqueous buffer to give a maximum final concentration of ≧30 mg/ml of the parent free-form of the compound. The suspension was equilibrated at 25° C. for 24 hours then the pH was measured and adjusted with either HCl or NaOH if it deviated by more than 0.3 units from the target pH. The suspension was then filtered through a glass fiber C filter. The filtrate was then diluted by an appropriate factor e.g. 101. Quantitation was by HPLC using method summarized in Table 5 with reference to a standard solution of approximately 0.25 mg/ml in DMSO. Different volumes of the standard, diluted and undiluted sample solutions were injected. The solubility was calculated using the peak areas determined by integration of the peak found at the same retention time as the principal peak in the standard injection.

Analysis was performed on an Agilent HP1100 series system equipped with a diode array detector and using ChemStation software. The following buffers were prepared to carry out the solubility analyses:

pH 1.2—made up by adding 85.0 mL of 0.2M HCl(aq) to 50 mL of 0.2M KCl(aq) and making up to 200 mL.
pH 4.5—made up by adding 2.99 g of NaC₂H₃O₂.3H₂O to 14.0 mL of 2N AcOH(aq) and making up to 1000 mL.
pH 7.4—made up adding 39.1 mL of 0.2M NaOH(aq) to 50.0 mL of a 0.2M KH₂PO₄ and making up to 200 mL.

TABLE 5
HPLC method parameters for solubility measurements.

| Type of method | Reverse phase with gradient elution | | |
|---|---|---|---|
| Column | Phenomenex Luna, C18 (2) 5 μm 50 × 4.6 mm | | |
| Column Temperature | 25 | | |
| (° C.) | | | |
| Standard Injections (μl) | 1, 2, 3, 5, 7, 10 | | |
| Test Injections (μl) | 1, 2, 3, 10, 20, 50 | | |
| Detection: Wavelength, | 260, 80 | | |
| Bandwidth (nm) | | | |
| Flow Rate (ml/min) | 2 | | |
| Phase A | 0.1% TFA in water | | |
| Phase B | 0.085% TFA in acetonitrile | | |
| Timetable | Time (min) | % Phase A | % Phase B |
| | 0.0 | 95 | 5 |
| | 1.0 | 80 | 20 |
| | 2.3 | 5 | 95 |
| | 3.3 | 5 | 95 |
| | 3.5 | 95 | 5 |
| | 4.4 | 95 | 5 |

### Nuclear Magnetic Resonance (¹H-NMR)

¹H-NMR spectra were collected on a Bruker 400 MHz instrument equipped with an autosampler and controlled by a DRX400 console. Automated experiments were acquired using ICON-NMR running with Topspin using the standard Bruker loaded experiments. For non-routine spectroscopy, data were acquired through the use of Topspin alone. Samples were prepared in DMSO-d6, unless otherwise stated. Off-line analysis was carried out using ACD SpecManager.

### Ion Chromatography (IC)

Data were collected on a Metrohm 861 Advanced Compact IC using IC Net software. Accurately weighed samples were prepared as stock solutions in an appropriate dissolving solution and diluted appropriately prior to testing. Quantification was achieved by comparison with standard solutions of known concentration of the ion being analyzed. The method can be summarized below:

| | |
|---|---|
| Type of method: | Anion exchange |
| Column: | Metrosep A Supp 5 - 250 (4.0 × 250 mm) |
| Column Temperature (° C.): | Ambient |
| Injection (μl): | 20 |
| Detection: | Conductivity detector |
| Flow Rate (ml/min): | 0.7 |
| Eluent: | 3.2 mM sodium carbonate, |
| | 1.0 mM sodium hydrogen carbonate in 5% |
| | aqueous acetone. |

### pKa Determination and Prediction.

Data were collected on a Sirius G1pKa instrument with a D-PAS attachment. Measurements were made at 25° C. in aqueous solution by UV and in methanol water mixtures by potentiometry. The titration media was ionic-strength adjusted (ISA) with 0.15 M KCl (aq). The values found in the methanol water mixtures were corrected to 0% co-solvent via a Yasuda-Shedlovsky extrapolation. The data were refined using Refinement Pro software v2.2. Prediction of pKa values was made using ACD/Labs software.

### Log P Determination and Prediction

Data were collected by potentiometric titration on a Sirius G1pKa instrument using three ratios of octanol:ionic-strength adjusted (ISA) water to generate Log P, Log Pion, and Log D values. The data were refined using Refinement Pro software. Prediction of Log P values was made using ACD/Labs software.

### Storage at Elevated Humidity

Samples were place on a glass slide and sealed in an airtight plastic container, along with an open beaker of saturated aqueous salt solution, and stored at a known regulated temperature. A saturated solution of NaCl was used to obtain a relative humidity of 75% at 40° C., whilst a saturated solution of potassium nitrate was used to provide 92% RH at 25° C.

### Example 1

### Characterization of Crystalline 4-(4-(imidazo[1,2-b]pyridazin-2-ylmethoxy)phenyl)-2,2-dimethyl-5-(pyridin-4-yl)furan-3(2H)-one

Two crystalline forms, Form 1 and 2, of 4-(4-(imidazo[1,2-b]pyridazin-2-ylmethoxy)phenyl)-2,2-dimethyl-5-(pyridin-4-yl)furan-3(2H)-one were determined by XRPD (see FIG. 2). The structure and high chemical purity were confirmed by ¹H-NMR and HPLC analyses.

The TGA and DSC thermograms of the crystalline Form 1 are shown in FIG. 5.

The property of the crystalline Form 2 is summarized in Table 6. Thermal analysis of Form 2 was performed, showing a weight loss of ˜1.3% w/w associated with an endotherm, likely to be a phase change during which water is lost (FIG. 8). A sharp melting endotherm at 185° C. was observed prior to decomposition starting at 275° C. The experiment was repeated under a stream of compressed air, which contained 20% v/v O₂. Based on the fact that the thermal plot did not change, it was concluded that oxidation does not occur.

TABLE 6
Characterization of Crystalline Form 2 of the Free Base.

| XRPD | Crystalline Form 2. |
|---|---|
| ¹H-NMR | Consistent with structure. |
| | 0.05 equivalents residual DCM. |
| Purity by HPLC | 99.0% (UV) |
| | Sum of total impurities greater than 0.1% = 0.3% (UV) |
| TGA + DSC | Weight loss of ~1.3% before 175° C. associated to broad |
| | endotherm with onset at 147° C., corresponding to loss of |
| | water and phase change. |
| | Melting endotherm with onset at 185° C., followed by |
| | decomposition from 275° C. |
| VT-XRPD | Form 2 converted to Form 1 on heating above 170° C., |
| | remaining as Form 1 on cooling to ambient. This |
| | resulting sample was reheated to the melt, and later |
| | re-crystallized as Form 1. |

Stability to elevated humidity

| | |
|---|---|
| Stability at 40° C./75% RH | No significant changes by XRPD or purity after 6 weeks |
| Stability at 25° C./96% RH | No significant changes by XRPD or purity after 6 weeks |

Stability to oxidation

| | |
|---|---|
| TGA and DSC under air | No changes with respect to experiments in N₂ |
| atmosphere | atmosphere. |
| | TGA at 2° C./min did not show any differences. |
| Stability at 60° C. in contact with | No significant changes by XRPD or HPLC analyses after |
| air | 6 weeks. |
| Stability at room conditions | No significant changes by XRPD or HPLC analyses after |
| (25° C. in contact with air) | 6 weeks. |

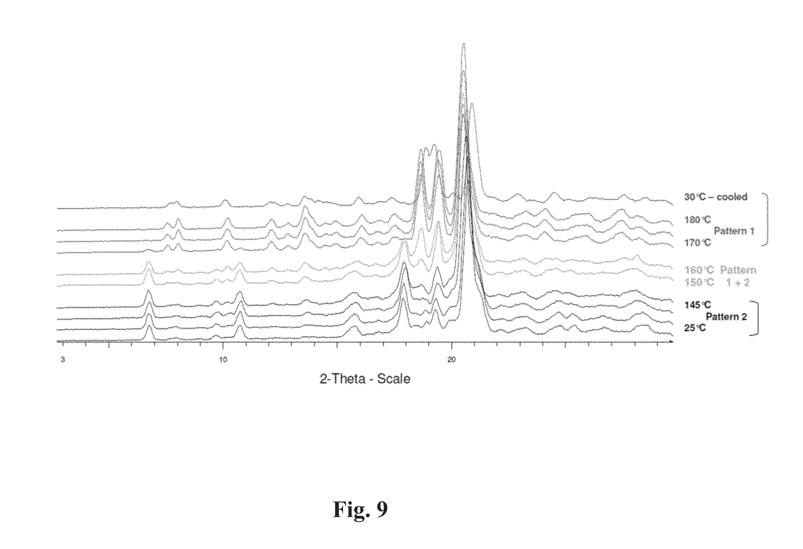

VT-XRPD was performed, showing that Form 2 converted to Form 1 on heating above 170° C., with the latter remaining on cooling to room temperature. The VT-XRPD spectrum are shown in FIG. 9.

### Example 2

### Thermodynamic Solubility

The thermodynamic solubility was determined for both Form 1 and Form 2 in deionized water and in buffers at pH 1.2, 4.5 and 7.4. Both Forms exhibited solubility in acidic conditions (between 30-40 mg/mL at pH 1.2). The results are summarized in Table 7. Very low solubility was observed for the experiments in deionized (DI) water, and pH 4.5 and 7.4 buffers.

TABLE 7
Thermodynamic solubility of Forms 1 and 2 of the Free Base.

| | | Thermodynamic | pH of saturated |
|---|---|---|---|
| | | solubility | unfiltered |
| Form | Solubility media | mg/mL | solution |
| Form 1 | DI water | 0.06 | 7.67 |
| | pH 1.2 | 32.00 | 1.40 |
| | pH 4.5 | 0.06 | 4.67 |
| | pH 7.4 | 0.05 | 7.46 |
| Form 2 | DI water | 0.04 | 7.23 |
| | pH 1.2 | 37.79 | 1.73 |
| | pH 4.5 | 0.05 | 4.65 |
| | pH 7.4 | 0.04 | 7.46 |

None of the salt formations in the initial screen produced a solid, even after addition of 500 μl of hexanes to the vessels. All the reactions were cooled to −18° C., which failed to yield any solid material. As such, the vessels were allowed to evaporate to dryness. Any solids produced were analyzed by XRPD.

### Example 2

### Stability Studies

The stability to elevated humidity and air oxidation was investigated for both Form 1 and Form 2 of the Free Base. Samples of both materials were stored at 40° C./75% RH, 25° C./96% RH, 60° C. in contact with air and ambient conditions (25° C. in contact with air) for six weeks, and analyses by XRPD, HPLC and ¹H-NMR (for oxidation study samples only) were carried out weekly. No significant changes were observed for either of the crystalline forms, by any of the techniques applied. XRPD analysis showed slight differences in crystallinity, probably due to sample preparation. The results are summarized in Tables 8-11.

TABLE 8
Stability study results for Form 2 (99% area % purity) at elevated humidity conditions.

| | | Week 1 | Week 2 | Week 3 | Week 4 | Week 5 | Week 6 |
|---|---|---|---|---|---|---|---|
| 25° C./96% | XRPD | No | No | No | No | No | No |
| RH | | significant | significant | significant | significant | significant | significant |
| | | changes | changes | changes | changes | changes | changes |
| | Purity | 99.3 | 98.9 | 98.9 | 98.9 | 98.0* | 98.9 |
| | (area %) | | | | | Slightly | |
| | | | | | | lower | |
| 40° C./75% | XRPD | No | Very slight | No | No | No | No |
| RH | | significant | differences | significant | significant | significant | significant |
| | | changes | could be due | changes | changes | changes | changes |
| | | | to sample | | | | |
| | | | preparation | | | | |
| | Purity | 99.5 | 99.1 | 99.2 | 99.1 | 98.8* | 98.9 |
| | (area %) | | | | | | |

*Purity week 5 samples run after 24 hours in solution.

TABLE 9
Stability study results for Form 1 (97% area % purity) at elevated humidity conditions.

| | | Week 1 | Week 2 | Week 3 | Week 4 | Week 5 | Week 6 |
|---|---|---|---|---|---|---|---|
| 25° C./96% | XRPD | No | No | No | No | No | No |
| RH | | significant | significant | significant | significant | significant | significant |
| | | changes | changes | changes | changes | changes | changes |
| | Purity | 96.6 | 95.5 | 96.5 | 95.9 | 91.8* | 95.5 |
| | (area %) | | | | | Slightly | |
| | | | | | | lower | |
| 40° C./75% | XRPD | No | No | No | No | No | No |
| RH | | significant | significant | significant | significant | significant | significant |
| | | changes | changes | changes | changes | changes | changes |
| | Purity | 96.9 | 96.3 | 96.5 | 95.6 | 95.6* | 96.8 |
| | (area %) | | | | Slightly | Slightly | |
| | | | | | lower | lower | |

*Purity week 5 samples run after 24 hours in solution.

TABLE 10
Stability study results for Form 2 (99% area %
purity) stored at 60° C. and room conditions.

| | | Week 1 | Week 2 | Week 3 | Week 4 | Week 5 | Week 6 |
|---|---|---|---|---|---|---|---|
| 60° C. | XRPD | No | No | No | No | No | No |
| | | significant | significant | significant | significant | significant | significant |
| | | changes | changes | changes | changes | changes | changes |
| | ¹H-NMR | No | No | No | Very weak | No | No |
| | | significant | significant | significant | | significant | significant |
| | | changes | changes | changes | | changes | changes |
| | Purity | 98.9 | 99.1 | n/d** | 99.0 | 98.6* | 99.0 |
| | (area %) | | | | | | |
| Room | XRPD | No | No | No | No | No | No |
| conditions | | significant | significant | significant | significant | significant | significant |
| | | changes | changes | changes | changes | changes | changes |
| | ¹H-NMR | No | No | No | No | No | No |
| | | significant | significant | significant | significant | significant | significant |
| | | changes | changes | changes | changes | changes | changes |
| | Purity | 99.2 | 98.6 | n/d** | 98.6 | 98.2* | 98.2 |
| | (area %) | | | | | | |

*Purity week 5 samples run after 24 hours in solution.

TABLE 11
Stability study results for Form 1 (97% area % purity) stored at 60° C. and room conditions.

| | | Week 1 | Week 2 | Week 3 | Week 4 | Week 5 | Week 6 |
|---|---|---|---|---|---|---|---|
| 60° C. | XRPD | No | No | No | No | Very low | Very low |
| | | significant | significant | significant | significant | crystallinity | crystallinity |
| | | changes | changes | chaises | changes | due to | due to |
| | | | | | | limited | limited |
| | | | | | | sample | sample |
| | ¹H-NMR | No | No | No | No | No | No |
| | | significant | significant | significant | significant | significant | significant |
| | | changes | changes | changes | changes | changes | changes |
| | Purity | 96.8 | 95.2 | 95.5 | 94.8 | 96.0* | 95.9 |
| | (area %) | | | | Slightly | | |
| | | | | | lower | | |
| Room | XRPD | No | No | No | No | No | No |
| conditions | | significant | significant | significant | significant | significant | significant |
| | | changes | changes | changes | changes | changes | changes |
| | ¹H-NMR | No | No | No | No | No | No |
| | | significant | significant | significant | significant | significant | significant |
| | | changes | changes | changes | changes | changes | changes |
| | Purity | 96.5 | 94.9 | 96.6 | 96.0 | 95.5* | 95.9 |
| | (area %) | | | | | | |

*Purity week 5 samples run after 24 hours in solution.

The Form 1 sample showed no significant changes after storage at 60° C. for 4 weeks and, no significant changes were observed by ¹H-NMR or HPLC after six weeks storage.

### Example 3

### Polymorphism Studies

### Slow Cooling and Evaporation Experiments.

4-(4-(imidazo[1,2-b]pyridazin-2-ylmethoxy)phenyl)-2,2-dimethyl-5-(pyridin-4-yl)furan-3(2H)-one (Form 2 or Form 1; ca. 10 mg per experiment) was weighed into vials, and the stated solvents were added, in increasing portions at 50° C. With each addition, the vials were allowed to shake for ˜20 minutes. Any solutions observed were allowed to cool to room temperature. Those experiments that remained as solutions were pin-holed to allow the solvent to slowly evaporate. Any suspensions observed were subjected to heat/cool cycles, between room temperature and 50° C., four hours at each condition.

### Form 2

Prism-shaped crystals were observed for most of the experiments. Most crystals presented enough size and quality and selected samples were submitted for single crystal structure determination, which confirmed the non-solvated nature of this crystalline form. Crystals were also ground in order to carry out XRPD analysis, which confirmed that all crystals analyzed were Form 2. For those experiments which remained in suspension, XRPD analysis was carried out after 24 hours cycling. No changes were observed, with all remaining as Form 2. These results are summarized in Table 12.

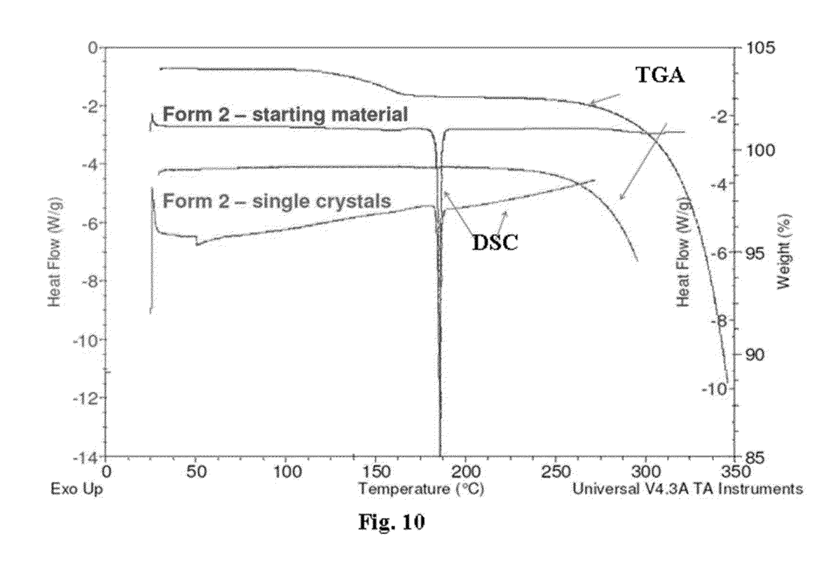

TGA and DSC analyses were performed on re-crystallized Form 2, to avoid the presence of water observed in the batch, which is not part of the crystal structure. There is no weight loss before decomposition by TGA, whereas DSC still shows the phase change endotherm as a very broad, weak event, albeit significant, with onset at 149° C. The results are shown in FIG. 10.

TABLE 12
Slow cooling and evaporation experiments of the crystalline Form 2 of the Free Base.

| | Solubility (volumes) | | | | | |
|---|---|---|---|---|---|---|
| Solvent | 10 | 20 | 50 | 100 | Procedure | XRPD |
| Methanol | Almost | ✓ | | | Cooling −> SCXRD | SCXRD - Form 2 |
| Ethanol | x | x | Almost | ✓ | Slow evaporation | Form 2 |
| IPA | x | x | Almost | Almost | Filtration + slow | Form 2 |
| | | | | | evaporation | |
| Acetonitrile | x | x | ✓ | | Cooling −> SCXRD | SCXRD - Form 2 |
| Acetone | x | x | ✓ | | Slow evaporation | Form 2 |
| MEK | x | Almost | ✓ | | Slow evaporation | Insufficient |
| | | | | | | material |
| MIBK | x | x | Almost | Almost | Filtration + slow | Insufficient |
| | | | | | evaporation | material |
| EtOAc | x | x | ✓ | | Cooling | Form 2 |
| IPAc | x | x | Almost | Almost | Filtration + slow | Insufficient |
| | | | | | evaporation | material |
| BuOAc | x | x | Almost | Almost | Filtration + slow | Insufficient |
| | | | | | evaporation | material |
| Cyclohexane | x | x | x | x | Heat/cool cycles | Form 2 |
| Heptane | x | x | x | x | Heat/cool cycles | Form 2 |
| Toluene | x | x | Almost | ✓ | Slow evaporation | Form 2 |
| DIPE | x | x | x | x | Heat/cool cycles | Form 2 |
| TBME | x | x | x | x | Heat/cool cycles | Insufficient |
| | | | | | | material |
| THF | ✓ | | | | Slow evaporation | Form 2 |
| 1-4-Dioxane | ✓ | | | | Slow evaporation | Form 2 |
| Nitromethane | Almost | ✓ | | | Slow evaporation | Form 2 |
| DCM | ✓ | | | | Slow evaporation | Insufficient |
| | | | | | | material |
| water | x | x | x | x | Heat/cool cycles | Form 2 |
| IPA/H₂O | ✓ | | | | Cooling | Form 2 |
| MEK/H₂O | ✓ | | | | Slow evaporation | Insufficient |
| | | | | | | material |
| THF/H₂O | ✓ | | | | Slow evaporation | Insufficient |
| | | | | | | material |
| MeCN/H₂O | Almost | ✓ | | | Cooling −> SCXRD | SCXRD - Form 2 |

✓ = fully soluble
X = not fully soluble

### Hot Stage Microscopy

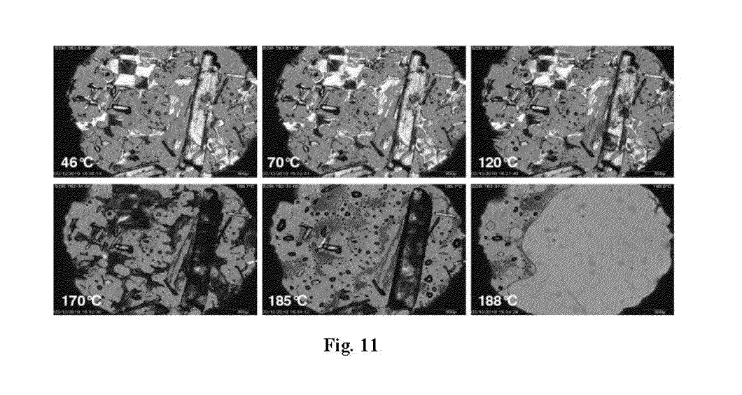

Hot Stage Microscopy was also performed on single crystals of Form 2. Evaporation of traces of solvents can be observed at low temperatures, with more crystals appearing. The sample then experienced a phase change, indicated by a change in the crystals color, between 120-170° C., followed by the melt from 184° C. The sample did not recrystallize on cooling. The color change is indicative of the form change from Form 2 to Form 1 (see FIG. 11). Based on this experiment, Form 1 single crystals were successfully prepared by heating Form 2 single crystals to 175° C., and the structure was determined.

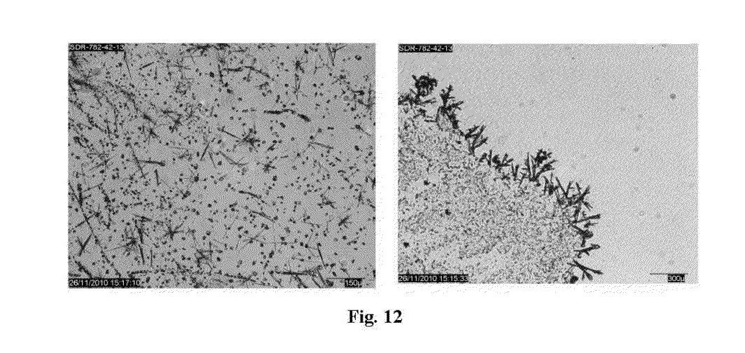

Prism-shaped crystals were observed for most of the experiments. These were ground in order to carry out XRPD analysis. The results confirmed that all crystallized solids were Form 2. Only the experiment from toluene produced smaller needle-shaped crystals of Form 1 by slow cooling. For those ‘slow cooling’ experiments shown in Table 13 which remained in suspension, XRPD analyses were carried out after 24 hours cycling between room temperature and 50° C. No changes were observed, with all samples remaining as Form 1. Slight differences were observed in the XRPD diffractograms due to the presence of large crystals and preferred orientation. The optical images of the crystalline Form 1 of the Free Base obtained by slow cooling from toluene solution are shown in FIG. 12.

TABLE 13
Slow cooling and evaporation experiments of
the crystalline Form 1 of the Free Base.

| | Solubility (volumes) | | | | | |
|---|---|---|---|---|---|---|
| Solvent | 10 | 20 | 50 | 100 | Procedure | XRPD |
| Methanol | x | ✓ | | | Cooling | Form 2 |
| Ethanol | x | x | ✓ | | Cooling | Form 2 |
| IPA | x | x | x | ✓ | Cooling | Form 2 |
| Acetonitrile | x | x | ✓ | | Cooling | Form 2 |
| Acetone | x | x | ✓ | | Cooling | Form 2 |
| MEK | x | x | ✓ | | Slow evaporation | Form 2 |
| MIBK | x | x | x | ✓ | Cooling | Form 2 |
| EtOAc | x | x | ✓ | | Cooling | Form 2 |
| IPAc | x | x | x | ✓ | Cooling | Form 2 |
| BuOAc | x | x | x | ✓ | Slow evaporation | Insufficient |
| | | | | | | material |
| Cyclohexane | x | x | x | x | Heat/cool cycles | Form 1 |
| Heptane | x | x | x | x | Heat/cool cycles | Form 1 |
| Toluene | x | x | ✓ | | Cooling | Form 1 |
| DIPE | x | x | x | x | Heat/cool cycles | Form 1 |
| TBME | x | x | x | x | Heat/cool cycles | Form 1 |
| THF | x | ✓ | | | Slow evaporation | Form 2 |
| 1-4-Dioxane | ✓ | | | | Slow evaporation | Form 2 |
| Nitromethane | ✓ | | | | Cooling | Form 2 |
| DCM | ✓ (RT) | | | | Slow evaporation | Form 2 |
| water | x | x | x | x | Heat/cool cycles | Form 1 |
| 1PA/H₂O | x | x | ✓ | | Cooling | Form 2 |
| MEK/H₂O | x | ✓ | | | Cooling | Form 2 |
| THF/H₂O | ✓ | | | | Slow evaporation | Form 2 |
| MeCN/H₂O | x | Almost | ✓ | | Slow evaporation | Form 2 |

✓ = fully soluble
X = not fully soluble

### Melt and Quench Cool

A DSC experiment was performed to assess the possibility of preparing amorphous material by melting and quench cooling. A sample of Form 1 (˜1 mg) was heated to 200° C. until it melted, then quickly cooled down to −80° C. and re-heated at 10° C./min. A weak glass transition was observed at 44° C., which was followed by an exothermic crystallization to Form 1 at 82° C. The crystalline Form 1 then melted at 184° C. This re-heating behavior indicated that the preparation of amorphous material had been successful.

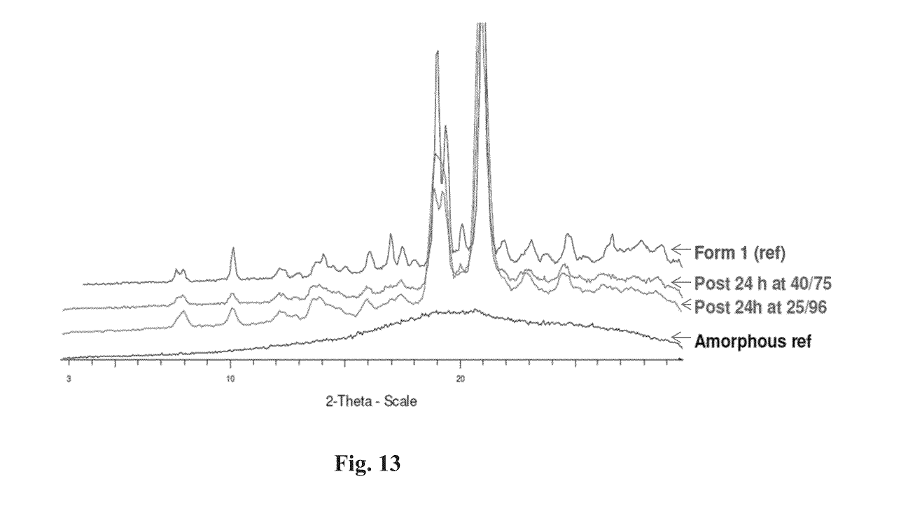

The experiment was repeated on a larger scale (batches of ˜60 mg) on the TGA instrument, and the resulting glasses were used as input materials for maturation experiments. The amorphous material converted to Form 1 on standing at room conditions (typically 25° C., 40% RH) as shown in FIG. 13. Samples were also stored at elevated humidity conditions for re-analysis by XRPD after 24 h. All solids displayed Form 1.

### Maturation Experiments

Amorphous material (ca. 10 mg per experiment) were weighed into vials and suspended in the solvents in which neither Form 1 or Form 2 dissolved during the slow cooling experiments. MEK was also used. The suspensions were subjected to heat/cool cycles between room temperature and 50° C., four hours at each condition, for two days.

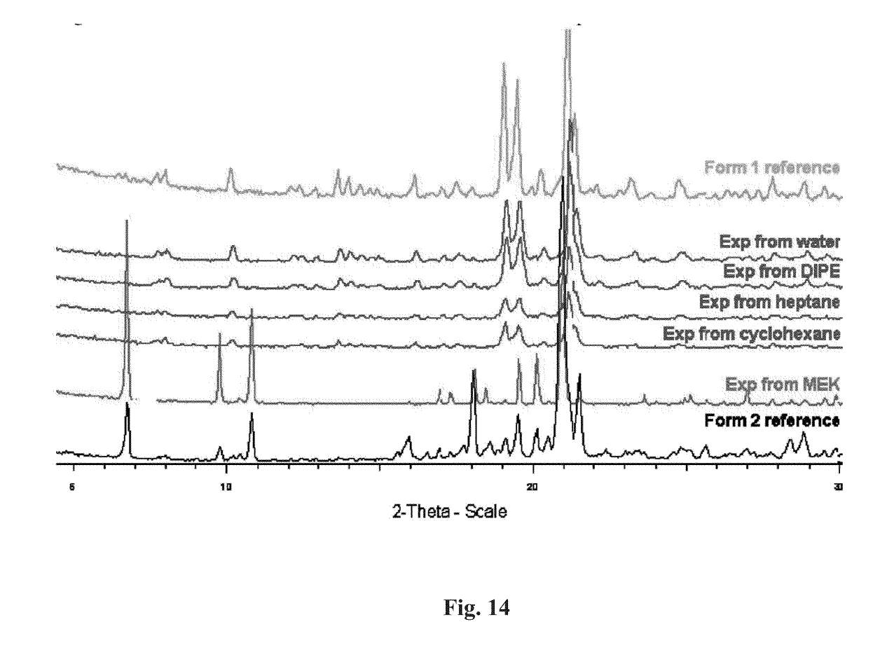

Crystalline solids were obtained from all experiments. Experiments in MEK produced Form 2, whereas the rest of the experiments produced Form 1. XRPD diffractograms are shown in FIG. 14.

### Example 4

### Stability of Relevant Polymorphs

### Determination of Transition Temperature

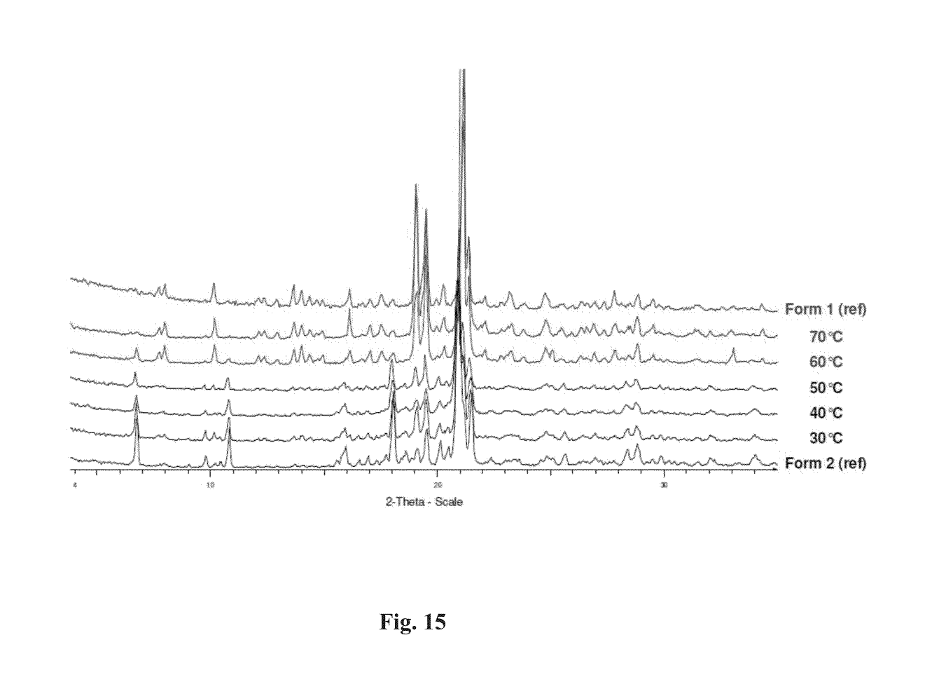

The transition temperature for the two crystalline forms was investigated by competitive slurry experiments. A mechanical mixture of both Form 1 and Form 2 was prepared and analyzed by XRPD for reference. The selected solvent for the experiments was IPA, given that both forms showed some solubility but not too high, and also because it has a moderate boiling point, making it suitable for the range of temperatures of interest. The mixture (ca. 20 mg) was suspended in IPA, and matured at different temperatures for 48 hours, in a range between 30 and 70° C. The resulting solids were analyzed by XRPD as shown in FIG. 15.

Completion was not reached during the maturation time at temperature below 50° C. However, enrichment in Form 2 was observed below this temperature, while at 60° C. or above Form 1 was observed. Thus, the transition temperature was established between 50 and 60° C., with Form 2 being stable below this temperature, and Form 1 being stable above.

### Example 5

### Single Crystal Experiments

A number of samples were submitted for single crystal X-Ray diffraction studies as shown in Table 14. The results for Forms 1 and 2 are shown in Tables 1 and 2, respectively.

TABLE 14
Samples submitted for single crystal X-Ray diffraction studies.

| Form | Experimental Reference |
|---|---|
| Form 1 | Slow cooling from solution |
| | in acetonitrile |
| Form 2 | Heating Form 2 single |
| | crystals to 175° C. |

### Form 1

The structure solution was obtained by direct methods, full-matrix least-squares refinement on F² with weighting w⁻¹=σ(Fₒ²)+(0.0520P)²+(0.0000P), where P=(Fₒ²+2F_{c}²)/3, anisotropic displacement parameters. Empirical absorption correction using spherical harmonics, implemented in SCALE3 ABSPACK scaling algorithm. Final wR²={Σ[w(Fₒ²−F_{c}²)²]/Σ[w(Fₒ²)²]^{1/2}=0.1403 for all data, conventional R₁=0.058 on F values of 4810 reflections with Fₒ>4σ(Fₒ), S=1.004 for all data and 563 parameters. Final Δ/σ(max) 0.000, Δ/σ(mean), 0.000. Final difference map between +0.204 and −0.266 e Å⁻³.

FIG. 3 shows a view of two molecules of Form 1 in the asymmetric unit from the crystal structure. Anisotropic atomic displacement ellipsoids for the non-hydrogen atoms are shown at the 50% probability level. Hydrogen atoms are displayed with an arbitrarily small radius. FIG. 4 shows a view of part of the crystal packing of Form 1 in the unit cell looking approximately down the [1,0,1] direction of the unit cell. For clarity, all hydrogen atoms other than the O—H and N—H have been removed.

### Form 2

The structure solution was obtained by direct methods, full-matrix least-squares refinement on F² with weighting w⁻¹=σ(Fₒ²)+(0.0500P)²+(0.6900P), where P=(Fₒ²+2F_{c}²)/3, anisotropic displacement parameters. Empirical absorption correction using spherical harmonics, implemented in SCALE3 ABSPACK scaling algorithm. Final wR²={Σ[w(Fₒ²−F_{c}²)²]/Σ[w(Fₒ²)²]^{1/2}=0.0944 for all data, conventional R₁=0.0357 on F values of 5913 reflections with Fₒ>4σ(Fₒ), S=1.005 for all data and 563 parameters. Final Δ/σ(max) 0.000, Δ/σ(mean), 0.000. Final difference map between +0.207 and −0.262 e Å⁻³.

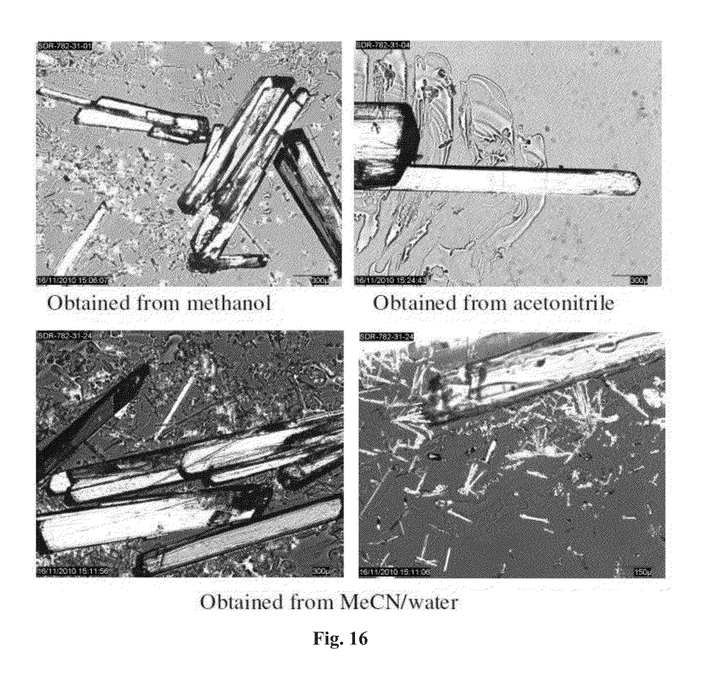

FIG. 6 shows a view of two molecules of Form 2 in the asymmetric unit from the crystal structure. Anisotropic atomic displacement ellipsoids for the non-hydrogen atoms are shown at the 50% probability level. Hydrogen atoms are displayed with an arbitrarily small radius. FIG. 7 shows a view of part of the crystal packing of Form 2 in the unit cell looking approximately down the [1,0,1] direction of the unit cell. For clarity, all hydrogen atoms other than the O—H and N—H have been removed. FIG. 16 shows the optical images of single crystals of the crystalline Form 2 of the Free Base.

A polymorphism study has been carried out. During this study, two enantiotropic, non-solvated crystalline forms have been identified, Form 1 and Form 2. A single crystal structure was obtained for each polymorph, thus confirming the non-solvated nature of both crystalline forms. The results of this study are shown in Table 15.

TABLE 15
Summary of properties of the Crystalline Form 1 and Form 2 of the Free Base.

| | Form 1 | Form 2 |
|---|---|---|
| Structure | Anhydrous | Anhydrous |
| | Single crystal structure | Single crystal structure |
| Thermal analysis | Melting point 185° C. | Endothermic phase transition to |
| | | Form 1 |
| | | at 145° C.(@10° C./min) |
| | | Weight loss of 1.2% w/w in the |
| | | batch corresponds to trapped water, |
| | | which is not part of the structure |
| Stability to humidity | No changes observed at elevated | No changes observed at elevated |
| | humidity and temperature | humidity and temperature |
| | conditions after 6 weeks | conditions after 6 weeks |
| Crystallization | Form 1 crystallized from cooling | Slow cooling yielded Form 2 |
| experiments | from toluene and by maturation of | in all occasions but one |
| | amorphous material in several | |
| | solvent systems | |

| | |
|---|---|
| Polymorphism studies | No other forms observed |
| Relative stability | Transition temperature between 50-60° C. |
| | Form 1 is stable above that Tₜᵣ, Form 2 is stable below |

The relative thermodynamic stability of the polymorphic pair Form 1/Form 2 was also investigated. Thermal analysis data for Form 2 showed an endothermic phase change at ˜145° C., suggesting that the system is enantiotropic. The transition temperature was established between 50-60° C. by competitive slurries of a mechanical mixture of Form 1 and Form 2 at different temperatures in IPA. Form 2 was the most stable form below the transition temperature while Form 1 was the thermodynamically stable form above it. A six-week stability study on both crystalline forms was performed. Samples of both Form 1 and Form 2 were stored at 40° C./75% RH, 25° C./96% RH, 60° C. and ambient conditions (25° C./40% RH), and analyzed by XRPD and HPLC during six weeks, with both materials showing no significant degradation after storage under any of those conditions. Despite the relatively lower stability of Form 1 compared with Form 2 at room temperature, no transformation of Form 1 into Form 2 has been observed in the solid state. Only in the presence of seeds (during slurring mixture of the two forms), did Form 1 transform to Form 2 at room temperature. Form 2 did not convert to Form 1 in the solid state at ambient conditions or upon storage at high humidity. This transformation only occurred upon heating above the phase-change temperature (145° C.).

### Example 6

### Salt Screen of the Free Base

### Chemical Purity of Form 1 by HPLC

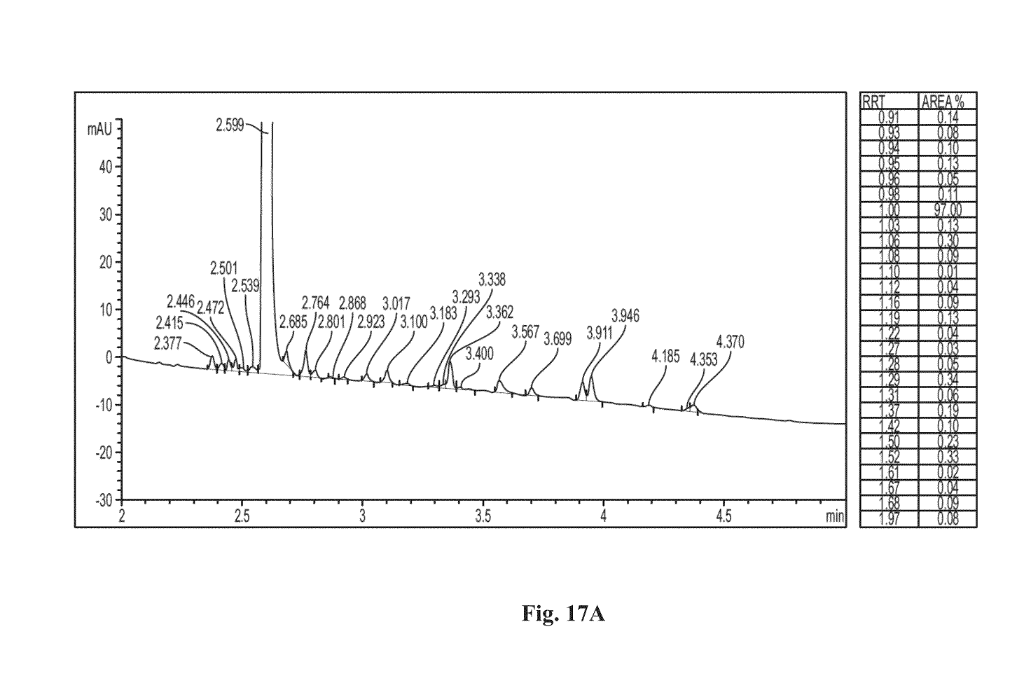

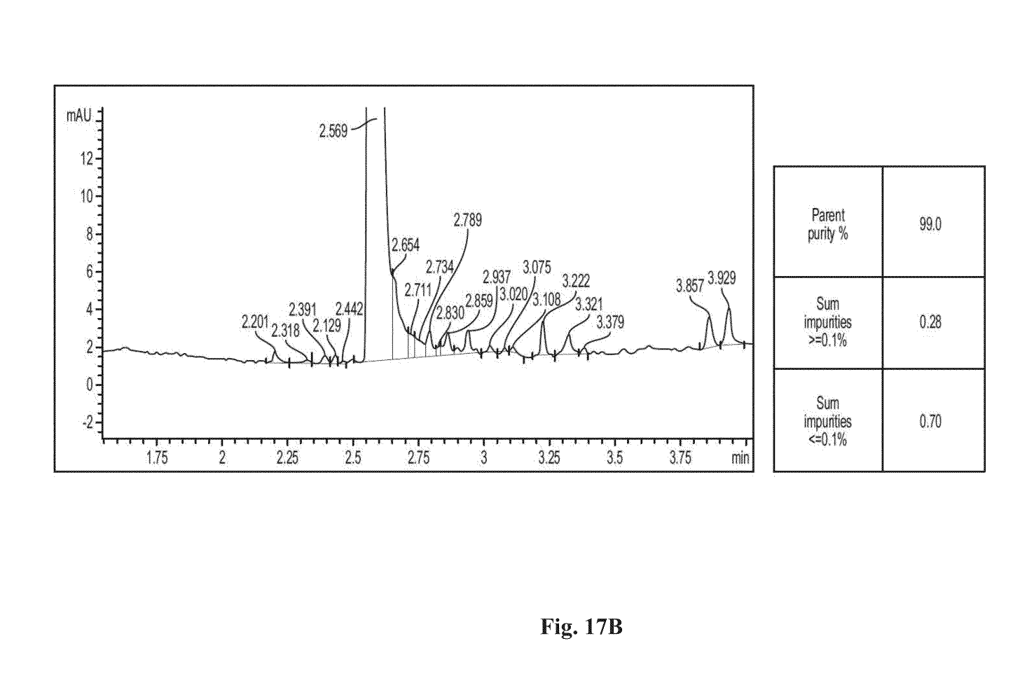

The chemical purity of crystalline Form 1 of the Free Base was determined by HPLC (FIG. 17).

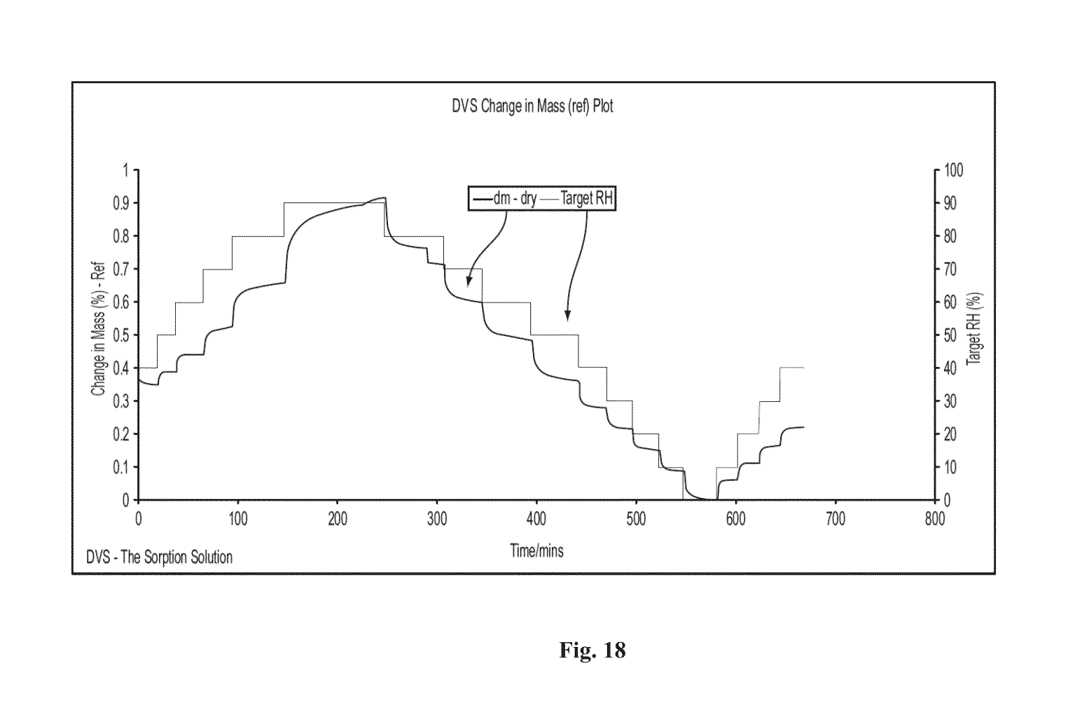

The GVS analysis of the crystalline Form 1 of the Free Base is shown in FIGS. 18 (kinetic plot) and 19 (isotherm plot). GVS analysis showed the material to be only slightly hygroscopic, taking up 0.9% of its mass in water upon raising the RH from 0% to 90%. No form change was noted by XRPD during this process. Storage of samples of the material at 25° C./92% RH and at 40° C./75% RH for seven days resulted in no significant change in the XRPD pattern, again indicating that no form change had occurred.

The Free Base possesses two basic centers, with respective pKa values of 3.70 and 2.90. These are significantly lower than the predicted values, impacting on the set of acids chosen in the later counter-ion screen. The log P for the compound was determined as 3.00, showing good agreement with the predicted value of 2.90. The predicted and measured pKa values for the two basic nitrogens in the Free Base are shown in Scheme 1 below.

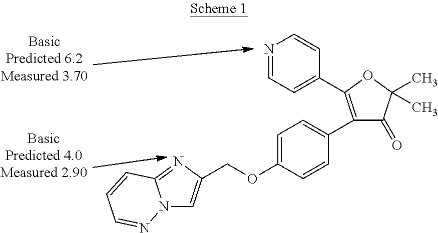 [SMILES: CC1(C)OC(c2ccncc2)=C(c2ccc(OCc3cn4ncccc4n3)cc2)C1=O]

### Salt Selection Studies

### Solvent Compatibility Evaluation

The Free Base was weighed into each of 24 HPLC vials and the relevant solvent added in portions until either a clear solution was obtained, or 1.0 mL total solvent had been added. The relevant acid was then added (1.0 eq), either as a 4 M solution in 1,4-dioxane (for HCl) or a 1M solution in THF (for ethanesulphonic acid). Any precipitation was noted and the vials were placed in an incubator cycling between ambient and 50° C. every four hours. After 17 hours incubation, any solids present were filtered off and analyzed by XRPD. These results are summarized in Table 16.

TABLE 16
Observations from solvent compatibility evaluation.

| | | Solubility | | | | HCI (A) | | Ethanesulphonic acid (B) | |
|---|---|---|---|---|---|---|---|---|---|
| | | 10 | 20 | 50 | 100 | Observations | | Observations | |
| Index | Solvent | vol | vol | vol | Vol | on addition | XRPD result | on addition | XRPD result |
| 1 | MeCN | X | X | ✓ | | no change | n/a | no change | n/a |
| 2 | IPA | X | X | X | X | no change | n′/a | no change | n/a |
| 3 | MEK | X | X | ✓ | | precipitation | partially crystalline | cloudy | partially |
| | | | | | | | new pattern | | crystalline new |
| | | | | | | | | | pattern |
| 4 | MIBK | X | X | X | X | precipitation | partially crystalline | cloudy | largely amorphous |
| | | | | | | | new pattern | | |
| 5 | DCM | X | ✓ | | | no change | partially crystalline | cloudy | n/a |
| | | | | | | | new pattern | | |
| 6 | THF | X | ✓ | | | precipitation | partially crystalline | cloudy | partially |
| | | | | | | | new pattern | | crystalline new |
| | | | | | | | | | pattern |
| 7 | EtOAc | X | X | X | ✓ | precipitation | partially crystalline | cloudy | n/a |
| | | | | | | | new pattern | | |
| 8 | IPAc | X | X | X | X | precipitation | partially crystalline | precipitation | largely amorphous |
| | | | | | | | new pattern | | |
| 9 | DIPE | X | X | X | X | precipitation | input material | lumpy solid | input material |
| 10 | TBME | X | X | X | X | no change | mixture | lumpy solid | partially |
| | | | | | | | | | crystalline new |
| | | | | | | | | | pattern |
| 11 | MeOH | X | X | ✓ | | no change | n/a | no change | n/a |
| 12 | EtOH | X | X | X | ✓ | no change | n/a | no change | n/a |

✓ = fully soluble
X = not fully soluble

As can be seen from Table 16, the Free Base exhibited good solubility in the more polar solvents, whilst failing to dissolve completely in 100 volumes of some of the less polar ones. Partially crystalline solids exhibiting novel XRPD patterns were isolated from several experiments in both the HCl and EtSO₃H sets, suggesting that salt formation occurs readily with these strong acids. Of the polar solvents in which the API was suitably soluble, MeOH and THF were selected for the screen.

Counter-Ion Screen with One Equivalent of Acid

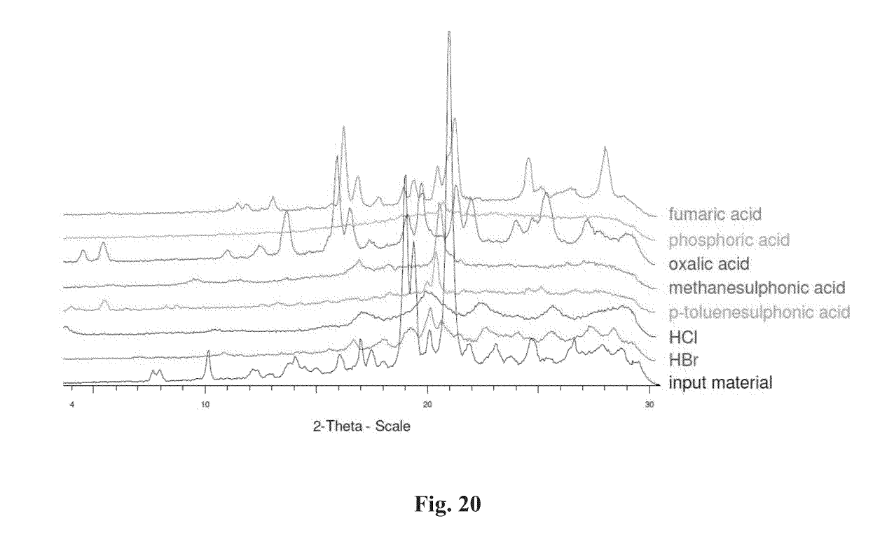

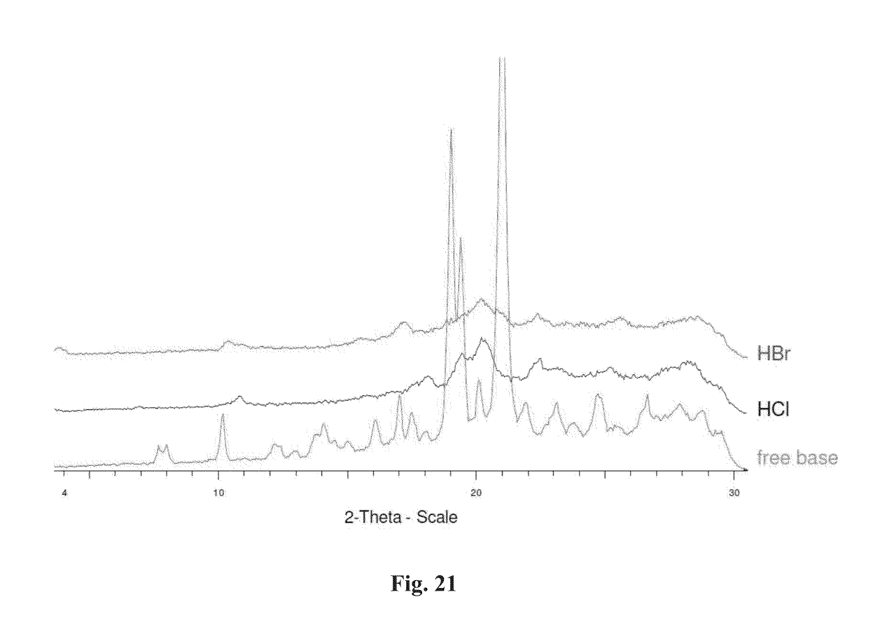

As the experimentally determined pKa values of the API proved to be less basic than predicted, to increase the chances of salt formation, the set of acids selected for the screen was biased towards those with a low first pKa. Free Base (ca. 50 mg) was accurately weighed into each of 34 reaction tubes and 1.5 mL of the relevant solvent (THF or MeOH) was added to each vessel. The tubes were heated, with stirring, to 60° C. and then 1.0 eq of the relevant acid added, in the form shown in Tables 17 and 18 below. Any precipitation was noted and then the tubes were cooled to 5° C. at ca. 10° C./hour and then stirred at 5° C. overnight. Any solids present were filtered off and analyzed by XRPD. To any tubes not containing solid was added enough TBME to cause cloudiness; the resulting mixtures were stirred at 5° C. overnight before any solid present was filtered and analyzed by XRPD. The XRPD results are shown in FIGS. 20 and 21.

TABLE 17
Counter-ion screen using one equivalent acid with the Free Base in THF.

| | | | Observations | Observations after | | XRPD |
|---|---|---|---|---|---|---|
| | Acid | Added as | on addition | cooling | Solid isolated | Result |
| 1 | Hydrobromic | 1M THF | orange/brown | orange/brown suspension, | after cooling | partially |
| | acid | soln | ppt | fine particles, hard to filter | | crystalline |
| 2 | Hydrochloric | 4M | orange/brown | orange/brown suspension, | after cooling | partially |
| | acid | dioxane | ppt | fine particles, hard to filter | | crystalline |
| | | soln | | | | |
| 3 | Sulphuric acid | 1M THF | yellow/brown | orange/brown suspension, | | |
| | | soln | ppt | deliquesced on filtering | | |
| 4 | p-Toluene | 1M EtOH | brown | brown solution | after anti- | partially |
| | sulphonic acid | | solution | | solvent | crystalline |
| | | | | | addition | |
| 5 | Methane | 1M THF | cloudy and | orange/brown suspension | after cooling | partially |
| | sulphonic acid | soln | oily solution | | | crystalline |
| 6 | Benzene | 1M THF | brown | brown solution | | |
| | sulphonic acid | soln | solution | | | |
| 7 | Oxalic acid | 1M THF | brown | brown solution | after anti- | highly |
| | | soln | solution | | solvent | crystalline |
| | | | | | addition | |
| 8 | L-Aspartic | solid | suspension of | cloudy orange brown | | |
| | acid | | added acid | mixture | | |
| 9 | Maleic acid | 1M THF | brown | brown solution | | |
| | | soln | solution | | | |
| 10 | Phosphoric | 1M THF | brown oil in a | brown oil. golden solution | after anti- | amorphous |
| | acid | soln | clear solution | | solvent | |
| | | | | | addition | |
| 11 | 1-Hydroxy-2- | 1M THF | brown | brown soln | | |
| | Naphthoic | soln | solution | | | |
| | acid | | | | | |
| 12 | Malonic acid | 1M THF | brown | brown soln | | |
| | | soln | solution | | | |
| 13 | L-Tartaric | 1M THF | brown | brown soln | | |
| | acid | soln | solution | | | |
| 14 | Fumaric acid | 0.5M | brown | brown soln, solid evolved | after cooling | highly |
| | | THF/MeOH | solution | on standing | | crystalline |
| | | soln | | | | |
| 15 | Citric acid | 1M THF | brown | brown soln | | |
| | | soln | solution | | | |
| 16 | L-Malic acid | 1M THF | brown | brown soln | | |
| | | soln | solution | | | |
| 17 | Acetic acid | 1M THF | brown | brown soln | | |
| | | soln | solution | | | |

TABLE 18
Counter-ion screen using one equivalent acid with the Free Base in MeOH.

| | | | Observations | Observations | | |
|---|---|---|---|---|---|---|
| Index | Acid | Added as | on addition | in morning | Solid isolated | XRPD Result |
| 1 | Hydrobromic | 1M THF | brown solution | brown solution | after anti-solvent | partially |
| | acid | soln | | | addition | crystalline |
| 2 | Hydrochloric | 4M | brown solution | brown solution | after anti-solvent | partially |
| | acid | dioxane | | | addition | crystalline |
| 3 | Sulphuric acid | 1M THF | brown solution | brown solution | | |
| | | soln | | | | |
| 4 | p-Toluene | 1M EtOH | brown solution | brown solution | | |
| | sulphonic acid | | | | | |
| 5 | Methane | 1M THF | brown solution | brown solution | | |
| | sulphonic acid | soln | | | | |
| 6 | Benzene | 1M THF | brown solution | brown solution | | |
| | sulphonic acid | soln | | | | |
| 7 | Oxalic acid | 1M THF | brown solution | brown solution | | |
| | | soln | | | | |
| 8 | L-Aspartic | solid | acid in | acid in | after anti-solvent | input acid |
| | acid | | suspension | suspension | addition | |
| 9 | Maleic acid | 1M THF | brown solution | brown solution | | |
| | | soln | | | | |
| 10 | Phosphoric | 1M THF | brown solution | brown solution | | |
| | acid | soln | | | | |
| 11 | 1-Hydroxy-2- | 1M THF | brown solution | brown solution | | |
| | Naphthoic | soln | | | | |
| | acid | | | | | |
| 12 | Malonic acid | 1M THF | brown solution | brown solution | | |
| | | soln | | | | |
| 13 | L-Tartaric acid | 1M THF | brown solution | brown solution | | |
| | | soln | | | | |
| 14 | Fumaric acid | 0.5M in | brown solution | brown solution | | |
| | | THF/MeOH | | | | |
| 15 | Citric acid | 1M THF | brown solution | brown solution | | |
| | | soln | | | | |
| 16 | L-Malic acid | 1M THF | brown solution | brown solution | | |
| | | soln | | | | |
| 17 | Acetic acid | 1M THF | brown solution | brown solution | | |
| | | soln | | | | |

Partially crystalline putative hydrobromide, hydrochloride, tosylate and mesylate salts were isolated from the experiments in THF, along with highly crystalline putative oxalate and fumarate salts. ¹H NMR analysis of these solids all showed shifts in resonances indicative of salt formation, except for the oxalate and fumarate salts. It is possible that these two solids are indeed salts, but dissociate readily upon dissolution in DMSO. This is supported by the observation that 1.0 eq of fumaric acid can be clearly seen in the spectrum. Interestingly, despite only 1 eq of acid being added, the experiment with p-toluenesulphonic acid clearly produced a solid with two molecules of acid per molecule of the Free Base. The solid isolated from the methanesulphonic acid experiment shows 1.7 eq of counter-ion present. The experiments using MeOH as solvent gave two hits, with poorly crystalline HCl and HBr salts being the only new solids isolated. ¹H-NMR analysis again provided evidence that salt formation had occurred, with significant shifts in resonances relative to the free base. The four isolated HCl and HBr salts all display similar XRPD patterns, suggesting that the hydrochloride and hydrobromide salts may be isostructural.

Counter-Ion Screen with Two Equivalents of Acid

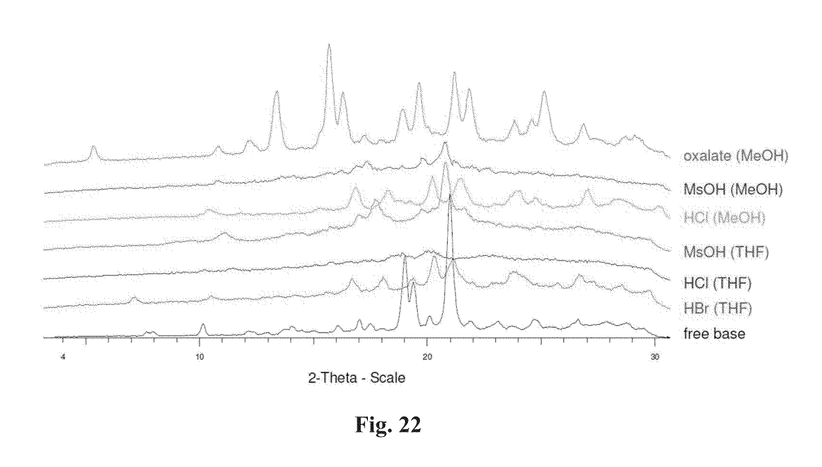

The seven strongest acids in the initial screening set were selected in order to give the best chance of forming bis-salts. Free Base (ca. 50 mg) was accurately weighed into each of 34 reaction tubes and 1.5 mL of the relevant solvent (THF or MeOH) added to each. The tubes were heated, with stirring, to 60° C. and then 2.0 eq of the relevant acid added, in the form shown in Table 19 below. Any precipitation was noted and then the tubes were cooled to 5° C. at ca. 10° C./hour and then stirred at 5° C. overnight. Any solids present were filtered off and analyzed by XRPD. To any tubes not containing solid was added enough TBME to cause cloudiness; the resulting mixtures were stirred at 5° C. overnight before any solid present was filtered and analyzed by XRPD (FIG. 22).

TABLE 19
Results of counter-ion screen of the Free Base using two equivalents of acid.

| | | | THF (A) | | | MeOH (B) | | |
|---|---|---|---|---|---|---|---|---|
| Details | | | Observations | Observations | | Observations | Observations | |
| Index | Acid | Added as | on addition | in morning | XRPD | on addition | in morning | XRPD |
| 1 | Hydrobromic | 1M | orange ppt | orange ppt | partially | brown | brown | |
| | acid | THF soln | | | crystalline | solution | solution | |
| 2 | Hydrochloric | 4M | orange ppt | orange ppt | partially | brown | brown | partially |
| | acid | dioxane | | | crystalline | solution | solution | crystalline |
| 3 | Sulphuric acid | 1M | yellow/ | yellow/ | deliquesced | brown | brown | |
| | | THF soln | orange ppt | orange ppt | | solution | solution | |
| 4 | p-Toluene | 1M | brown | brown | | brown | brown | |
| | sulphonic acid | EtOH | solution | solution | | solution | solution | |
| 5 | Methane | 1M | yellow/ | yellow/ | partially | brown | brown | partially |
| | sulphonic acid | THF soln | orange ppt | orange ppt | crystalline | solution | solution | crystalline |
| 6 | Benzene | 1M | brown | brown | | brown | brown | |
| | sulphonic acid | THF soln | solution | solution | | solution | solution | |
| 7 | Oxalic acid | 1M | brown | brown | | brown | brown | crystalline |
| | | THF soln | solution | solution | | solution | solution | |

The seven isolated solids all showed the same diffraction patterns as their counterparts isolated in the screen with one equivalent of acid, confirming that that particular screen had a tendency to form bis-salts. Interestingly, the tosylate salt observed in the mono-screen was not formed during this set of experiments.

### Large Scale Synthesis of Salts of the Free Base

Certain salts of the Free Base were synthesized in a larger scale. The properties of these salts were investigated.

The synthesis of these salts were carried out according to the general procedure as follows: Free Base (ca. 100 mg) was weighed into each of five glass vials. EtOAc (2.0 ml) was then added to each vial, followed by 2.1 eq of the relevant acid, in the same form as used in the screen (as the fumarate is known to be a mono salt, only 1 eq was added). The vials were each seeded with ca. 2 mg of solid isolated from the screen. Vials were then placed in an incubator and cycled between ambient and 50° C. for 72 h. Solids were then isolated by filtration, dried under vacuum overnight and characterized as detailed in the following sections.

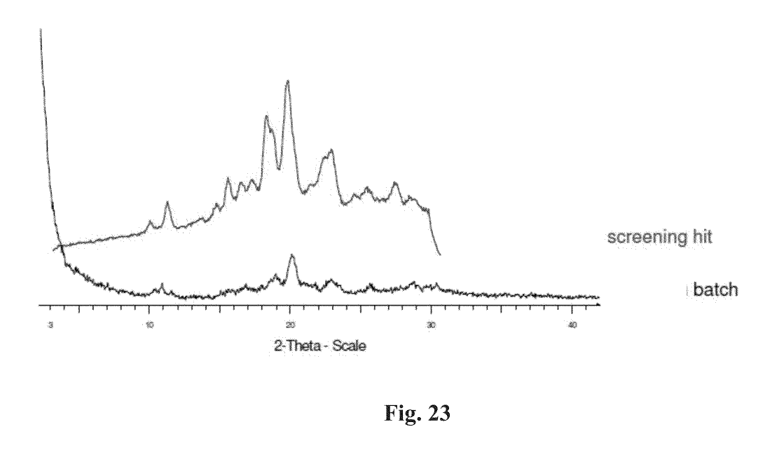

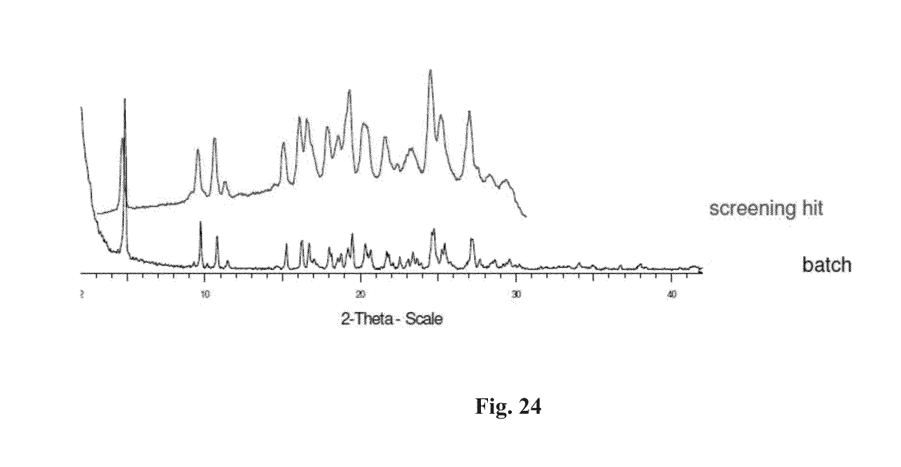

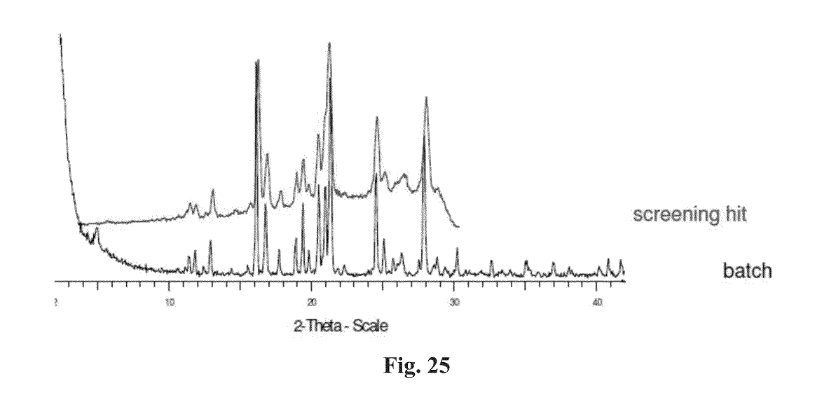

The XRPD spectrum of bis-HBr salt of the Free Base is shown in FIG. 23. The XRPD spectrum of bis-Tosylate salt of the Free Base is shown in FIG. 24. The XRPD spectrum of mono-Fumarate salt of the Free Base is shown in FIG. 25. Other salts such as bis-HCl salt and bis-mesylate salt were synthesized as well.

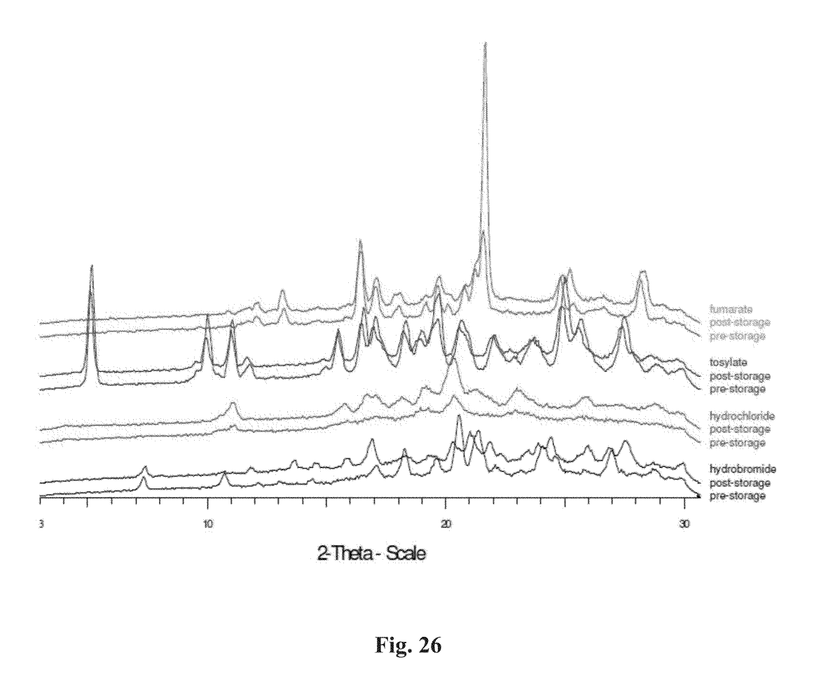

The stability of various salts formed was investigated by using XRPD. The results are shown in FIG. 26. The mesylate was observed to have deliquesced after 2 days storage at 40° C./75% RH, so no XRPD analysis was performed.

All five of the investigated salts (bis-HBr salt, bis-Tosylate salt, of mono-Fumarate salt, bis-HCl salt and bis-mesylate salt) produced material of the same crystalline form identified during the screen and no degradation was noted in the HPLC traces of any of the isolated salts. The mesylate salt was observed to deliquesce upon storage at high RH. The bis-hydrobromide salt contains ca. 0.2 eq of THF as evidenced by its ¹H-NMR spectrum, although it appears that this could be easily removed by drying at elevated temperature. The bis-tosylate salt shows clear bis-stoichiometry and appears to exist as a monohydrated form; a 2.46% mass loss in the TGA corresponding to one equivalent of water, with the preceding 1.18% loss likely attributable to surface bound water. The mesylate salt is poorly crystalline and appears to contain 2.2 eq of acid per molecule of API and, like the bis-tosylate, appears to exist in a monohydrated form. Of the solids isolated, the mono-fumarate displays the best solid form properties, being highly crystalline and exhibiting clear mono stoichiometry and excellent thermal stability.

### Solubility of the Salts

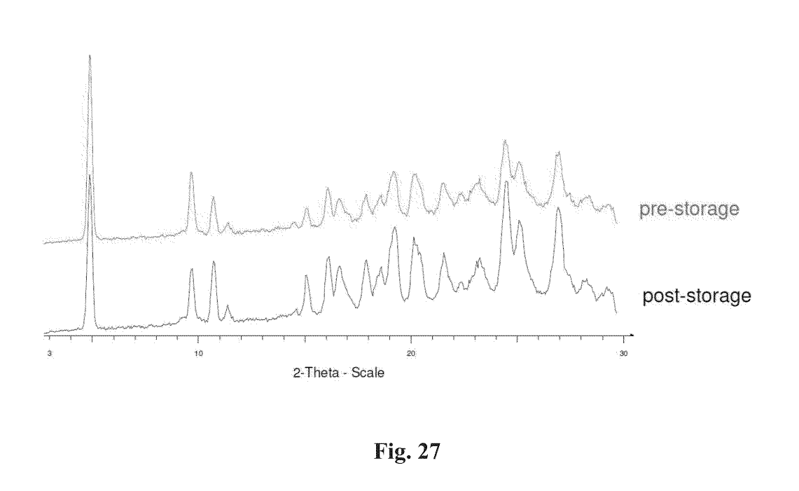

The solubility of the formed salts was then investigated. The solubility of the Free Base bis-tosylate salt is shown in Table 20. The stability of the Free Base bis-tosylate salt is shown in FIG. 27.

TABLE 20
Solubility profile of the Free Base bis-
tosylate and comparison with the Free Base.

| | | | pH of | |
|---|---|---|---|---|
| | | Solubility | unfiltered | |
| | | (mg mL free form | saturated | |
| Species | pH point | equivalent) | solution | Appearance |
| bis- | unadjusted | 29.07 | 2.00 | Residual Oil (Dark orange) |
| tosylate | aqueous | | | |
| bis- | 1.2 | 32.29 | 1.21 | Residual Solid- Orange |
| tosylate | | | | Solution |
| bis- | 4.5 | 0.05 | 4.52 | Residual Solid- Brown Solid |
| tosylate | | | | |
| bis- | 7.4 | 0.05 | 7.40 | Residual Solid- Brown Solid |
| tosylate | | | | |
| free base | unadjusted | 0.06 | 6.19 | Solid on Surface/Residual |
| | aqueous | | | Solid |
| free base | 1.2 | 12.19 | 1.24 | Orange Solution- Very fine |
| | | | | Suspension |
| free base | 4.5 | 0.09 | 4.71 | Residual Solid- Brown Solid |
| free base | 7.4 | 0.05 | 7.44 | Residual Solid- Brown Solid |

The Free Base bis-tosylate salt is confirmed as a monohydrated bis-p-toluenesulphonic acid salt. The material exhibits high crystallinity and is assessed as 99.0% pure using a generic HPLC method, meaning that the salt formation process has improved the purity above the 97.0% observed for the free base. Storage at 40° C./75% RH for a period of one week produced no form change. The salt exhibits an endotherm at onset 119° C. This could represent a melt or could be associated with dehydration. Below this temperature VT-XRPD showed no change in form, despite water being lost from the structure. This suggests that this crystalline form may correspond to a channel hydrate, i.e., the water of hydration is loosely bound and can be removed and replaced without disrupting the crystal lattice. This hypothesis is supported by the GVS data which shows completely reversible 3.1% water uptake over the 0% to 90% RH range, with no accompanying form change. Aqueous solubility is vastly improved over the free base, although in buffered solution, as expected, no significant difference is observed. The bis-tosylate is likely more soluble than the free base primarily because it lowers the aqueous pH while the free base does not. Both species are significantly more soluble at low pH. Dissolution rate experiments would likely provide a better handle on the advantage of the bis-tosylate over the free base in this regard.

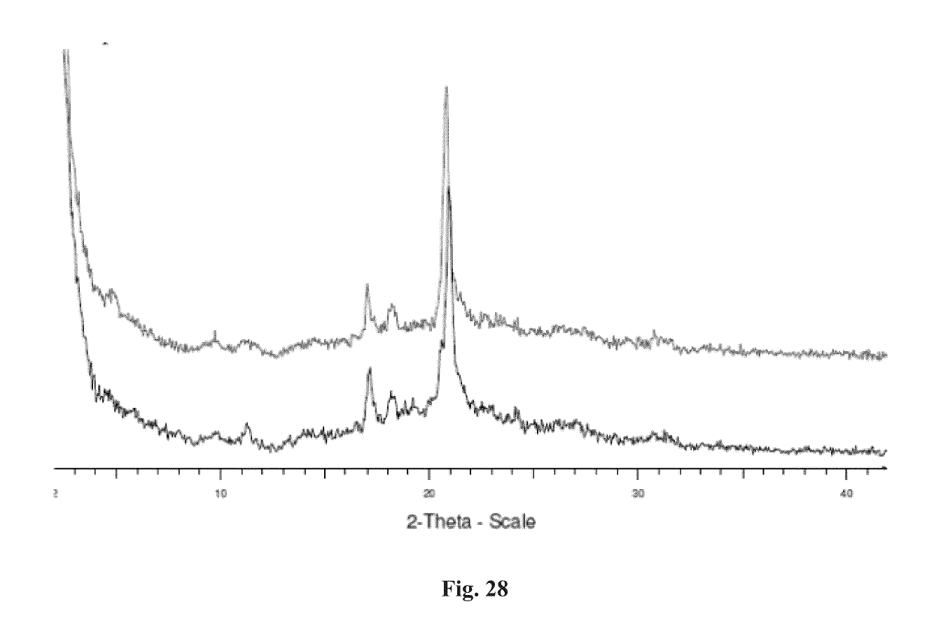

The bis-mesylate salt of the Free Base was also synthesized, which was shown to have poor crystallinity (FIG. 28).

The isolated bis-mesylate salt is of poor crystallinity and judging by the presence of two endotherms in the DSC thermogram, tentatively identified as melts as the onset temperatures do not vary with heating rate, exists as a mixture of two forms.

### Polymorphism Studies of the Salts

The isolated bis-mesylate salt and bis-tosylate salt were further investigated. These two salts were converted to amorphous states by heating samples of each to 180° C. until melted and then rapidly cooling down to 5° C., generating a glassy amorphous solid in each case. Mostly amorphous free base was generated by dissolving ca. 500 mg of the Free Base in 10 mL of DCM and rapidly removing the solvent using a rotary evaporator.

The relevant amorphous solid (ca. 10 mg) was weighed into each of 36 HPLC vials and 100 μL of the relevant solvent was added to each. The vials were placed in an incubator for 17 h, cycling the temperature between ambient and 50° C. every four hours. Solids present were then filtered off and analyzed by XRPD (FIG. 28). The results of polymorph assessment are summarized in Table 21. Here, “new pattern (2)” is the crystalline Form 2 as described above.

TABLE 21
Results of polymorph assessment on Free Base bis-tosylate salt, bis-mesylate salt, and Free Base.

| | | tosylate (A) | | mesylate (B) | | free base (C) | |
|---|---|---|---|---|---|---|---|
| Index | Solvent | Observations | XRPD | Observations | XRPD | Observations | XRPD |
| 1 | MeCN | brown solution | | small amount of | amorphous | yellow/brown | form one |
| | | | | yellow solid | | suspension | |
| 2 | MeCN + | brown solution | | brown suspension | partially | yellow/brown | form one |
| | 1% water | | | | crystalline | suspension | |
| 3 | IPA | yellow/brown | form one | brown solution | | yellow/brown | form one |
| | | suspension | | | | suspension | |
| 4 | MEK | yellow/brown | new | gum | | yellow/brown | new |
| | | suspension | pattern | | | suspension | pattern |
| | | | (2) | | | | (2) |
| 5 | MEK + | yellow/brown | form one | yellow/brown | | yellow brown | new |
| | 1% water | suspension | | gum | | suspension | pattern |
| | | | | | | | (2) |
| 6 | MIBK | yellow/brown | form one | yellow/brown | | yellow/brown | form one |
| | | suspension | | gum | | suspension | |
| 7 | DCM | brown solution | | brown solution | | brown solution | |
| 8 | THF | yellow/brown | form one | brown oil/gum | | brown solution | |
| | | suspension | | | | | |
| 9 | EtOAc | brown solid | form one | yellow/brown | | yellow/brown | form one |
| | | | | gum | | suspension | |
| 10 | TBME | brown solid | new | brown gum | | brown | form one |
| | | | pattern | | | suspension | |
| | | | (3) | | | | |
| 11 | MeOH | brown solution | | brown solution | | brown solution | |
| 12 | MeOH + | brown solution | | brown solution | | brown solution | |
| | 1% water | | | | | | |

The bis-tosylate salt showed some evidence of polymorphism, with different XRPD patterns observed for material isolated from MEK and TBME. These patterns appear to exhibit many of the same peaks as the input material, but with extra peaks added, thus suggesting that the material now exists as a mixture of forms. It is possible that full conversion to another form may occur if the maturations were left on for longer. Overall, evidence was obtained for the existence of two polymorphic forms in addition to the form first isolated. The maturation of the mesylate yielded only one solid with any crystallinity. This was isolated from the MeCN+1% water experiment and whilst exhibiting the same pattern as the input material the crystallinity was markedly improved.

What is claimed is:

1. Crystalline 4-(4-(imidazo[1,2-b]pyridazin-2-ylmethoxy)phenyl)-2,2-dimethyl-5-(pyridin-4-yl)furan-3(2H)-one, having a peak position at about 6.7, 10.8, 15.8, 18.0, 19.4, 20.2, 21.1, 21.5, or 28.8 degrees 2-theta in an x-ray powder diffraction pattern obtained using Cu-Kα radiation.

2. The crystalline 4-(4-(imidazo[1,2-b]pyridazin-2-ylmethoxy)phenyl)-2,2-dimethyl-5-(pyridin-4-yl)furan-3(2H)-one of claim 1, having a peak position at about 6.7, 10.8, 18.0, 19.4, 21.1, or 21.5 degrees 2-theta in an x-ray powder diffraction pattern obtained using Cu-Kα radiation.

3. The crystalline 4-(4-(imidazo[1,2-b]pyridazin-2-ylmethoxy)phenyl)-2,2-dimethyl-5-(pyridin-4-yl)furan-3(2H)-one of claim 1, characterized by the X-ray powder diffractogram substantially as shown in FIG. 2, Form 2, when measured at room temperature using Cu-Kα radiation.

4. The crystalline 4-(4-(imidazo[1,2-b]pyridazin-2-ylmethoxy)phenyl)-2,2-dimethyl-5-(pyridin-4-yl)furan-3(2H)-one of claim 1, characterized by respective lattice parameters, a, b, and c of about 11.9 Å, 18.0 Å, and 19.4 Å, respectively, and β of about 102.8° in the monoclinic crystal system P2₁ space group, when measured with Cu-Kα radiation at about 100 K.

5. The crystalline 4-(4-(imidazo[1,2-b]pyridazin-2-ylmethoxy)phenyl)-2,2-dimethyl-5-(pyridin-4-yl)furan-3(2H)-one of claim 1, which is at least about 95% or at least about 97% chemically pure.

6. The crystalline 4-(4-(imidazo[1,2-b]pyridazin-2-ylmethoxy)phenyl)-2,2-dimethyl-5-(pyridin-4-yl)furan-3(2H)-one of claim 1, which is at least about 99% chemically pure.

7. The crystalline 4-(4-(imidazo[1,2-b]pyridazin-2-ylmethoxy)phenyl)-2,2-dimethyl-5-(pyridin-4-yl)furan-3(2H)-one of claim 5 or 6, wherein the purity is determined by HPLC.

8. The crystalline 4-(4-(imidazo[1,2-b]pyridazin-2-ylmethoxy)phenyl)-2,2-dimethyl-5-(pyridin-4-yl)furan-3(2H)-one of claim 1, having an endothermic onset at about 140-165° C. in a differential scanning calorimetry (DSC) profile.

9. The crystalline 4-(4-(imidazo[1,2-b]pyridazin-2-ylmethoxy)phenyl)-2,2-dimethyl-5-(pyridin-4-yl)furan-3(2H)-one of claim 8, having an endothermic onset at about 145° C. in a differential scanning calorimetry (DSC) profile.

10. The crystalline 4-(4-(imidazo[1,2-b]pyridazin-2-ylmethoxy)phenyl)-2,2-dimethyl-5-(pyridin-4-yl)furan-3(2H)-one of claim 1, having an endothermic onset at about 185° C. in a differential scanning calorimetry (DSC) profile.

11. The crystalline 4-(4-(imidazo[1,2-b]pyridazin-2-ylmethoxy)phenyl)-2,2-dimethyl-5-(pyridin-4-yl)furan-3(2H)-one of claim 8, characterized by the differential scanning calorimetry (DSC) profile substantially as shown in FIG. 8.

12. The crystalline 4-(4-(imidazo[1,2-b]pyridazin-2-ylmethoxy)phenyl)-2,2-dimethyl-5-(pyridin-4-yl)furan-3(2H)-one of claim 8, which is stable at room temperature under air for at least about 4, 6, 8, 10, 12, or 20 weeks.

13. Crystalline 4-(4-(imidazo[1,2-b]pyridazin-2-ylmethoxy)phenyl)-2,2-dimethyl-5-(pyridin-4-yl)furan-3(2H)-one, having a peak position at about 7.9, 8.0, 10.2, 13.7, 14.0, 16.2, 17.6, 19.1, 19.3, 21.2, or 21.4 degrees 2-theta in an x-ray powder diffraction pattern obtained using Cu-Kα radiation.

14. The crystalline 4-(4-(imidazo[1,2-b]pyridazin-2-ylmethoxy)phenyl)-2,2-dimethyl-5-(pyridin-4-yl)furan-3(2H)-one of claim 13, having a peak position at about 19.1, 19.3, 21.2, or 21.4 degrees 2-theta in an x-ray powder diffraction pattern obtained using Cu-Kα radiation.

15. The crystalline 4-(4-(imidazo[1,2-b]pyridazin-2-ylmethoxy)phenyl)-2,2-dimethyl-5-(pyridin-4-yl)furan-3(2H)-one of claim 13, characterized by the X-ray powder diffractogram substantially as shown in FIG. 2, Form 1, when measured at room temperature using Cu-Kα radiation.

16. The crystalline 4-(4-(imidazo[1,2-b]pyridazin-2-ylmethoxy)phenyl)-2,2-dimethyl-5-(pyridin-4-yl)furan-3(2H)-one of claim 13, characterized by respective lattice parameters, a, b, and c of about 12.2 Å, 27.4 Å, and 12.4 Å, respectively, and β of about 96.7° in the monoclinic crystal system P2₁ space group, when measured with Cu-Kα radiation at about 120 K.

17. The crystalline 4-(4-(imidazo[1,2-b]pyridazin-2-ylmethoxy)phenyl)-2,2-dimethyl-5-(pyridin-4-yl)furan-3(2H)-one of claim 13, which is at least about 95% or at least about 97% chemically pure.

18. The crystalline 4-(4-(imidazo[1,2-b]pyridazin-2-ylmethoxy)phenyl)-2,2-dimethyl-5-(pyridin-4-yl)furan-3(2H)-one of claim 13, which is at least about 99% chemically pure.

19. The crystalline 4-(4-(imidazo[1,2-b]pyridazin-2-ylmethoxy)phenyl)-2,2-dimethyl-5-(pyridin-4-yl)furan-3(2H)-one of claim 17 or 18, wherein the purity is determined by HPLC.

20. The crystalline 4-(4-(imidazo[1,2-b]pyridazin-2-ylmethoxy)phenyl)-2,2-dimethyl-5-(pyridin-4-yl)furan-3(2H)-one of claim 13, having an endothermic onset at about 180-190° C. in a differential scanning calorimetry (DSC) profile.

21. The crystalline 4-(4-(imidazo[1,2-b]pyridazin-2-ylmethoxy)phenyl)-2,2-dimethyl-5-(pyridin-4-yl)furan-3(2H)-one of claim 20, having an endothermic onset at about 185-186° C. in a differential scanning calorimetry (DSC) profile.

22. The crystalline 4-(4-(imidazo[1,2-b]pyridazin-2-ylmethoxy)phenyl)-2,2-dimethyl-5-(pyridin-4-yl)furan-3(2H)-one of claim 13, having a melting point of about 185-186° C.

23. The crystalline 4-(4-(imidazo[1,2-b]pyridazin-2-ylmethoxy)phenyl)-2,2-dimethyl-5-(pyridin-4-yl)furan-3(2H)-one of claim 20, characterized by the differential scanning calorimetry (DSC) profile substantially as shown in FIG. 5.

24. The crystalline 4-(4-(imidazo[1,2-b]pyridazin-2-ylmethoxy)phenyl)-2,2-dimethyl-5-(pyridin-4-yl)furan-3(2H)-one of claim 13, which is stable at room temperature under air for at least about 4, 6, 8, 10, 12, or 20 weeks.

25. A pharmaceutical composition comprising the crystalline 4-(4-(imidazo[1,2-b]pyridazin-2-ylmethoxy)phenyl)-2,2-dimethyl-5-(pyridin-4-yl)furan-3(2H)-one of any one of claims 1-23 and a pharmaceutically acceptable excipient.

26. The pharmaceutical composition of claim 25, wherein the crystalline 4-(4-(imidazo[1,2-b]pyridazin-2-ylmethoxy)phenyl)-2,2-dimethyl-5-(pyridin-4-yl)furan-3(2H)-one is present in a therapeutically effective amount.

27. The pharmaceutical composition of claim 25 or 26 formulated for oral administration.

28. The pharmaceutical composition of claim 27 in the form of a unit dosage.

29. The pharmaceutical composition of claim 27 in the form of a tablet, a capsule or a powder.

30. The pharmaceutical composition of claim 29 in the form of a tablet.

31. A method of treating CNS disorders, eating disorders, obesity, compulsive gambling, sexual disorders, narcolepsy, sleep disorders, diabetes, metabolic syndrome, schizophrenia, schizo-affective conditions, Huntington's disease, bipolar disorders, dystonic conditions and tardive dyskinesia, or for use in smoking cessation treatment in a patient comprising administering to the patient in need thereof an effective amount of the crystalline 4-(4-(imidazo[1,2-b]pyridazin-2-ylmethoxy)phenyl)-2,2-dimethyl-5-(pyridin-4-yl)furan-3(2H)-one of any one of claims 1-24.

32. The method of claim 31, wherein the patient is a mammal.

33. The method of claim 32, wherein the mammal is a human.

34. The method of any one of claims 31-33, wherein the crystalline 4-(4-(imidazo[1,2-b]pyridazin-2-ylmethoxy)phenyl)-2,2-dimethyl-5-(pyridin-4-yl)furan-3(2H)-one is orally administered.

35. The method of claim 34, wherein the crystalline 4-(4-(imidazo[1,2-b]pyridazin-2-ylmethoxy)phenyl)-2,2-dimethyl-5-(pyridin-4-yl)furan-3(2H)-one is administered once or twice daily.

36. The method of any one of claims 31-35, wherein the crystalline 4-(4-(imidazo[1,2-b]pyridazin-2-ylmethoxy)phenyl)-2,2-dimethyl-5-(pyridin-4-yl)furan-3(2H)-one is administered as a tablet or a capsule.

37. The method of any one of claims 31-36, wherein the method is for treating schizophrenia.

38. The method of any one of claims 31-36, wherein the method is for treating Huntington's disease.
